(19) United States
(12) Patent Application Publication
(10) Pub. No.: US 2025/0032443 A1
(43) Pub. Date: Jan. 30, 2025

(54) TREATMENT OF MYOTONIC DISORDERS

(71) Applicant: University of Rochester, Rochester, NY (US)

(72) Inventors: John D. Lueck, Rochester, NY (US); Lily Cisco, Rochester, NY (US); Charles A. Thornton, Rochester, NY (US)

(73) Assignee: University of Rochester, Rochester, NY (US)

(21) Appl. No.: 18/708,955

(22) PCT Filed: Nov. 10, 2022
(86) PCT No.: PCT/US22/79593

§ 371 Date: May 9, 2024

Related U.S. Application Data
(60) Provisional application No. 63/374,378, filed on Sep. 2, 2022.
(60) Provisional application No. 63/277,816, filed on Nov. 10, 2021.

(51) Int. Cl. A61K 31/277; A61K 31/4418; A61K 31/554; A61K 45/06; A61P 21/00

(52) U.S. Cl. CPC A61K 31/277; A61K 31/4418; A61K 31/554; A61K 45/06; A61P 21/00

(57) ABSTRACT

This disclosure relates to the unexpected discovery that agents capable of inhibiting calcium channels, such as calcium channel blockers, are effective in treating or alleviating the symptoms of myotonic disorders. In patients who have been diagnosed with the condition, the methods of this disclosure can, e.g., reverse or inhibit the worsening of symptoms related to such condition and/or prevent development of new symptoms.

### CROSS-REFERENCE TO RELATED APPLICATIONS

This application claims priority under 35 U.S.C. § 119(e) to U.S. Provisional Patent Application No. 63/277,816, filed Nov. 10, 2021 and U.S. Provisional Patent Application No. 63/374,378, filed Sep. 2, 2022. The foregoing applications are incorporated by reference herein in their entirety.

### STATEMENT REGARDING FEDERALLY SPONSORED RESEARCH

This invention was made with government support under UL1TR002001 awarded by the National Institutes of Health. The government has certain rights in the invention.

### FIELD OF THE INVENTION

This invention relates to methods and agents for treatment of myotonic disorders.

### BACKGROUND OF THE INVENTION

Myotonic disorders are a group of genetic diseases characterized by the presence of myotonia, which is defined as failure of muscle relaxation after activation. The presentation of these disorders can range from asymptomatic electrical myotonia, as seen in some forms of myotonia congenita (MC), to severe disability with muscle weakness, cardiac conduction defects, and other systemic features as in myotonic dystrophy type I (DM1). The most prevalent form of muscular dystrophy in adults, DM1 is estimated to affect more than 100,000 people in the U.S., many of whom experience decades of progressive disability, with a median survival of 55 years of age. Due to the debilitating and progressive nature of these disorders, including myotonic dystrophy, resulting disability and loss of quality of life in patients, significant cost of supportive care, and the lack of an ultimate cure, there is a pressing need for new and effective means to treat myotonic disorders.

### SUMMARY OF THE INVENTION

This disclosure addresses the need mentioned above in a number of aspects. In one aspect, the disclosure provides a method of treating a myotonic disorder in a subject in need thereof. In some embodiments, the method comprises administering to the subject a therapeutically effective amount of an agent (e.g., a calcium channel blocker, such as a use-dependent calcium channel blocker) that inhibits a calcium channel in a cell of the subject. In some embodiments, the agent comprises a calcium channel blocker, a stereoisomer thereof, a derivative thereof or a pharmaceutically acceptable salt thereof, or a combination thereof.

In some embodiments, the calcium channel is a Cav1.1 calcium channel. In some embodiments, the subject is a mammal, e.g., a human.

In some embodiments, the agent is selected from verapamil, amlodipine, diltiazem, nifedipine, a stereoisomer, a derivative thereof or a pharmaceutically acceptable salt thereof, and a combination thereof.

In some embodiments, the myotonic disorder is myotonic dystrophy, autosomal dominant or recessive myotonia congenita, or sodium channel myotonia. In some embodiments, the myotonic dystrophy is myotonic dystrophy type 1 (DM1) or myotonic dystrophy type 2 (DM2).

In some embodiments, the myotonic dystrophy is associated with abnormal alternative splicing of RNA encoding CIC-1 chloride channel or Cav1.1 calcium channel. In some embodiments, the myotonia congenita is Becker or Thomsen myotonia congenita. In some embodiments, the sodium channel myotonia is paramyotonia congentia, hyperkalemic periodic paralysis, or potassium-aggravated myotonia. In some embodiments, the subject has one or more mutations in SCN4A, Clcn1, or Cac1ns. In some embodiments, the subject has alternative splice isoforms of Clcn1 or Cac1ns.

In some embodiments, the agent is administered to the subject at one or more doses of from about 0.01 to about 100 mg/kg of body weight of the subject. In some embodiments, the agent is administered at one or more doses of from about 1 to about 50 mg/kg of body weight of the subject. In some embodiments, the agent is administered at one or more doses of from about 8 to about 16 mg/kg of body weight of the subject.

In some embodiments, one or more doses of the agent are administered at least every 1 day, 3 days, 5 days, 1 week, 2 weeks, 3 weeks, or 4 weeks.

In some embodiments, the agent is administered to the subject intratumorally, intravenously, subcutaneously, intraosseously, orally, transdermally, sublingually, in sustained release, in controlled release, in delayed release, or as a suppository.

In some embodiments, the method further comprises administering to the subject an additional therapeutic agent or therapy. In some embodiments, the additional therapeutic agent is selected from ranolazine mexiletine, flecainide, tocainide, phenytoin, carbamazepine, lamotrigine, and a combination thereof. In some embodiments, the additional therapeutic agent or therapy is administered to the subject before, after, or concomitantly with the agent.

In some embodiments, the treatment produces a therapeutic effect selected from reduced myotonia, reduced body weight loss, improved survival, improved muscle function, improved time of righting reflex, improved respiratory function, and improved diaphragm strength. In some embodiments, the treatment results in at least 50% improvement in myotonia, body weight loss, survival, muscle function, time of righting reflex, respiratory function, or diaphragm strength as compared to an untreated subject.

In another aspect, this disclosure also provides use of an agent that inhibits a calcium channel in the manufacture of a medicament for treating a myotonic disorder in a subject in need thereof. In some embodiments, the agent is selected from verapamil, amlodipine, diltiazem, nifedipine, a stereoisomer thereof, a derivative thereof or a pharmaceutically acceptable salt thereof, and a combination thereof. In some embodiments, the calcium channel is a Cav1.1 calcium channel. In some embodiments, the myotonic disorder is myotonic dystrophy, autosomal dominant or recessive myotonia congenita, or sodium channel myotonia.

In yet another aspect, this disclosure further provides a pharmaceutical composition, e.g., for treating a myotonic disorder in a subject in need thereof. In some embodiments, the pharmaceutical composition comprises: (a) a calcium channel blocker selected from verapamil, amlodipine, diltiazem, nifedipine, and a combination thereof, and (b) one or more agents selected from ranolazine mexiletine, flecainide, tocainide, phenytoin, carbamazepine, and lamotrigine.

Also within the scope of this disclosure is a kit comprising the pharmaceutical composition described herein.

The details of one or more embodiments of the disclosure are set forth in the description below. Other features, objectives, and advantages of the disclosure will be apparent from the description and from the claims.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, 1E, and 1F are a set of graphs showing that Ca_{V}1.1^{Δe29} and ClC-1^{-/-} alleles exhibit synthetic lethality, and result in significantly reduced body weight and severe muscle weakness in mice. FIG. 1A shows the results Kaplan-Meier survival analysis of WT (n=10), Caᵥ1.1^{Δe29/+} (n=13), Caᵥ1.1^{Δe29} (n=15), ClC-1^{-/-} (n=40), Ca^{v}1.1^{Δe29/+}/ClC-1^{-/-} (n=16), and Caᵥ1.1^{Δe29/Δe29}/ClC-1^{-/-} (n=21). FIG. 1B shows the results of weekly body weight analysis. FIG. 1C shows percent body weight change from weaning at 10 weeks. FIG. 1D shows representative specific force traces, and FIG. 1E shows average peak specific force elicited by 150 Hz (500 ms) tetanic stimulation of isolated EDL muscles from 10-wk mice. FIG. 1F shows average frequency dependence of specific force generation, elicited from isolated EDL muscle from 10-wk mice. Symbols, open circles, individual mice; bars and closed circles, means±SEM.

FIGS. 2A, 2B, 2C, 2D, 2E, and 2F are a set of graphs showing that verapamil treatment improves survival, body weight, and muscle function in Caᵥ1.1^{Δe29}/ClC-1^{-/-} mice. FIG. 2A shows the results of the Kaplan-Meier survival analysis of WT+vehicle (n=10), WT+200 mg/kg/day verapamil (n=10), Caᵥ1.1^{Δe29}/ClC-1^{-/-} (n=37), and Caᵥ1.1^{Δe29/Δe29}/ClC-1^{-/-}+verapamil (n=9). Verapamil is dosed in mouse nutrition/hydration food cups. FIG. 2B shows the results of body weight analysis. FIGS. 2C and 2E show representative specific force traces elicited by twitch (left) and 150 Hz (500 ms) tetanic (right) stimulation of EDL muscle isolated from 10-wk (FIG. 2C) and 20-wk (FIG. 2E) mice. FIGS. 2D and 2F show a plot of average stimulation frequency dependence of specific force generation from isolated EDL muscles at 10 weeks (FIG. 2D) and 20 weeks (FIG. 2F) of age in the indicated genotype and treatment groups. Symbols, closed circles, means±SEM.

FIGS. 3A, 3B, and 3C are a set of graphs showing that verapamil treatment significantly improves the time of righting reflex in ClC-1^{-/-} and Caᵥ1.1^{Δe29}/ClC-1^{-/-} mice. FIG. 3A shows the results of weekly time of righting reflex analysis of Caᵥ1.1^{Δe29}/ClC-1^{-/-}, ClC-1^{-/-}, Caᵥ1.1^{Δe29}, WT, Caᵥ1.1^{Δe29}/ClC-1^{-/-}+verapamil and WT+verapamil mice, FIG. 3B shows average time of righting reflex in vehicle and verapamil treated mice at 10- and 20-week of age. Untreated Caᵥ1.1^{Δe29}/ClC-1^{-/-} mice do not survive to 20-week of age; therefore, the last recording before death is documented. Box notes Q1, median, and Q3; whiskers show minimum and maximum. FIG. 3C shows paired before (light circles) an after (dark circles) 2-wk verapamil treatment of ClC-1^{-/-} mice at 100 mg/kg/day (left) and 200 mg/kg/day (right) dosing in nutrition/hydration food cups. Symbols, closed circles, individual mice; open circles, means±SEM.

FIGS. 4A, 4B, 4C, 4D, 4E, 4F, and 4G are a set of graphs showing that Verapamil treatment significantly improves respiratory function and diaphragm strength in Caᵥ1.1^{Δe29}/ClC-1^{-/-} mice. Whole-body plethysmography for 10-wk (FIGS. 4A and 4C) and 20-wk (FIGS. 4B and 4D) old mice in the indicated genotype and treatment groups. FIGS. 4A and 4B show the frequency of respiration (breaths/min), and FIGS. 4C and 4D show tidal volume of respiration (mL). FIG. 4E shows representative tetanic (150 Hz, 500 ms) specific force traces from diaphragm strips isolated from 10-wk (left) and 20-wk (right) old mice in the indicated genotypes and treatment groups. FIGS. 4F and 4G show plots of average stimulation frequency dependence of specific force generated from diaphragm strips isolated from 10-wk (FIG. 4F) and 20-wk (FIG. 4G) old mice in the indicated genotypes and treatment groups. Symbols, open circles, individual mice; bars and closed circles, means±SEM. Note: Untreated Caᵥ1.1^{Δe29}/ClC-1^{-/-} mice do not survive to 20-week of age.

FIGS. 5A, 5B, 5C, and 5D are a set of graphs showing that Caᵥ1.1^{Δe29}/ClC-1^{-/-} muscle exhibits severe transient weakness that is significantly improved by the addition of verapamil. FIGS. 5A and 5C show normalized representative force traces of the first 15 tetani (100 Hz, 500 ms) separated by four seconds, recorded ex vivo from EDLs isolated from 6-wk ClC-1^{-/-} and Caᵥ1.1^{Δe29}/ClC-1^{-/-} mice in the absence (FIG. 5A) and presence (FIG. 5C) of 20 μM verapamil added to the bath. FIGS. 5B and 5D show plots of the average peak tetanic EDL forces normalized to the initial stimulus, elicited by 44 subsequent 100 Hz, 500 ms tetanic stimulations separated by four seconds from 6-wk WT (n=4), ClC-1^{-/-} (n=4), Caᵥ1.1^{Δe29} (n=4) and Caᵥ1.1^{Δe29}/ClC-1^{-/-}, n=4) mice in the absence (FIG. 5B) and presence (FIG. 5D) of 20 μM verapamil added to the bath for ClC-1^{-/-} (n=4) and Caᵥ1.1^{Δe29}/CC-1^{-/-}, n=4) EDLs. Dashed lines in FIG. 5D represent average data presented in FIG. 5B as a reference for pre-treatment. Symbols, closed circles, mean±SEM.

FIGS. 6A, 6B, 6C, 6D, 6E, 6F, 6G, and 6H are a set of graphs showing that verapamil significantly reduces myotonia in both Caᵥ1.1^{Δe29}/ClC-1^{-/-} and ClC-1^{-/-} mouse muscle. FIGS. 6A and 6C show normalized representative specific force traces of the first (left) and third (right) tetani (150 Hz, 500 ms) from EDLs isolated from 6-wk WT and Caᵥ1.1^{Δe29} mice in the absence (FIG. 6A) and presence (FIG. 6C) of 20 mM verapamil added to the bath. Dashed lines represent accumulated force. FIGS. 6B and 6D show average normalized integration of force for WT and Caᵥ1.1^{Δe29} EDLs across 3 tetanic stimulations (150 Hz, 500 ms) in the absence (FIG. 6B) and presence (FIG. 6D) of 20 mM verapamil added to the bath. FIGS. 6E and 6G show normalized representative specific force traces of the first (left) and third (right) tetani (150 Hz, 500 ms) from EDLs isolated from 6-wk ClC-1^{-/-} and Caᵥ1.1^{Δe29}/ClC-1^{-/-} mice in the absence (FIG. 6E) and presence (FIG. 6G) of 20 mM verapamil added to the bath. Dashed lines represent accumulated force. FIGS. 6F and 6H show average integration of force for ClC-1^{-/-} and Caᵥ1.1^{Δe29}/ClC-1^{-/-} EDLs across 3 tetanic stimulations (150 Hz, 500 ms) in the absence (FIG. 6B) and presence (FIG. 6D) of 20 mM verapamil added to the bath. Symbols, open circles, individual mice; bars, mean±SEM. Contralateral EDLs were used for each untreated and treated experiment. All traces plotted with the same scale of time and normalized force for comparison.

FIGS. 7A and 7B are a set of graphs showing that long term verapamil oral feeding of Caᵥ1.1^{Δe29}/ClC-1^{-/-} mice results in significant rescue of EDL muscle force generation. Average peak specific force elicited using a 150 Hz (500 ms) tetanus stimulus of EDLs isolated from 10-wk (FIG. 7A) and 20-wk (FIG. 7B) mice of indicated genotype and treatment. Symbol, open circles, individual mice; bars, mean±SEM.

FIGS. 8A and 8B are a set of graphs showing that long term verapamil oral feeding of Caᵥ1.1^{Δe29}/ClC-1^{-/-} mice results in significant rescue of diaphragm muscle force generation. Average peak specific force elicited using a 150 Hz (500 ms) tetanus stimulus of diaphragm strips isolated from 10-wk (FIG. 8A) and 20-wk (FIG. 8B) mice of indicated genotype and treatment. Symbol, open circles, individual mice; bars, mean±SEM.

FIGS. 9A, 9B, 9C, and 9D are a set of graphs showing the results of quantification and statistical analysis of fiber type distribution of tibialis anterior muscle. Quantification of type IIb (FIG. 9A), type Ix (FIG. 9B), type IIa (FIG. 9C), and type I (FIG. 9D) fibers for 10-wk (left) and 20-wk (right) tibialis anterior (n=5/group) are shown.

FIGS. 10A, 10B, 10C, and 10D are a set of graphs showing the results of quantification and statistical analysis of fiber type distribution of diaphragm muscle. Quantification of type IIb (FIG. 10A), type IIx (FIG. 10B), type IIa (FIG. 10C), and type I (FIG. 10D) fibers for 10-wk (left) and 20-wk (right) diaphragm (n=5/group).

FIGS. 11A, 11B, and 11C are a set of diagrams showing that heterozygous and homozygous Caᵥ1.1^{Δe29} mice exhibit similar Caᵥ1.1 voltage-dependence and peak current densities in flexor digitorum brevis muscle. FIG. 11A shows representative current density traces from whole cell patch clamp of flexor digitorum brevis fibers isolated from 4-week WT, Caᵥ1.1^{Δe29/+} (dashed), and Caᵥ1.1^{Δe29/Δe29} (solid) mice at 0 mV (top), +20 mV (middle) and +40 mV (bottom). FIG. 11B shows a plot of average current-voltage relationship of Caᵥ1.1 activity measured in WT, Caᵥ1.1^{Δe29/+} (circles, dashed), and Caᵥ1.1^{Δe29/Δe29} (circle, solid line) flexor digitorum brevis fibers isolated from 4-wk mice. FIG. 11C shows RT-PCR products of Caᵥ1.1 RNA isolated from tibialis anterior from 10-wk mice. PCR amplifications are from exons 27 to 31 of Cacalns cDNA.

FIGS. 12A, 12B, 12C, and 12D are a set of graphs showing that Caᵥ1.1^{Δe29}/ClC-1^{-/-} muscle exhibits severe transient weakness that is significantly improved by the addition of verapamil (not normalized to the first peak force). FIGS. 12A and 12C show representative specific force traces of the first 15 tetani (100 Hz, 500 ms) separated by four seconds, recorded ex vivo from EDLs isolated from 6-wk ClC-1^{-/-} and Caᵥ1.1^{Δe29}/ClC-1^{-/-} mice in the absence (FIG. 12A) and presence (FIG. 12C) of 20 μM verapamil added to the bath. FIGS. 12B and 12D show a plot of the average peak tetanic EDL, elicited by 44 subsequent 100 Hz, 500 ms tetanic stimulations separated by four seconds from 6-wk WT (n=4), ClC-1^{-/-} (n=4), Caᵥ1.1^{Δe29} (n=4) and Caᵥ1.1^{Δe29}/ClC-1^{-/-} n=4) mice in the absence (FIG. 12B) and (FIG. 12D) presence of 20 μM verapamil added to the bath for ClC-1^{-/-} (n=4) and Caᵥ1.1^{Δe29}/ClC-1^{-/-} n=4) EDLs. Dashed lines in FIG. 12D represent average data presented in FIG. 12B as a reference for pre-treatment. Symbols, closed circles, mean±SEM.

FIGS. 13A, 13B, 13C, 13D, 13E, 13F, 13G, and 13H are a set of graphs showing that verapamil treatment does not reduce peak contraction force of WT, Caᵥ1.1^{Δe29}, ClC-1^{-/-} and Caᵥ1.1^{Δe29}/ClC-1^{-/-} mouse muscle. FIGS. 13A, 13C, 13E, and 13G show representative traces of the first (left) and third (right) tetani (150 Hz, 500 ms) in WT (FIG. 13A), Caᵥ1.1^{Δe29} (FIG. 13C), ClC-1^{-/-} (FIG. 13E), and Caᵥ1.1^{Δe29}/ClC-1^{-/-} (FIG. 13G) EDLs in the absence and presence of 20 mM verapamil. Treatment is depicted in legends. FIGS. 13B, 13D, 13F, and 13H show average specific force for WT (FIG. 13B), Caᵥ1.1^{Δe29} (FIG. 13D), ClC-1^{-/-} (FIG. 13F), and Caᵥ1.1^{Δe29}/ClC-1^{-/-} (FIG. 13H) EDLs across 3 tetanic stimulations in the absence and presence of 20 mM verapamil. Treatment is depicted in legends. Symbols, open circles, individual mice; bars, mean, and SEM.

FIGS. 14A and 14B are a set of graphs showing that verapamil significantly reduces myotonia in both Caᵥ1.1^{Δe29}/ClC-1^{-/-} and ClC-1^{-/-} mouse muscle. FIG. 14A shows representative trace of normalized specific force generation of the first (left) and third (right) tetani (150 Hz, 500 ms) in ClC-1^{-/-} (solid) and Caᵥ1.1^{Δe29}/ClC-1^{-/-} (sold) EDL in the absence of 20 μM verapamil. Dashed lines represent accumulated force. FIG. 14B shows representative trace of the first (left) and third (right) tetani (150 Hz, 500 ms) in ClC-1^{-/-} (solid) and Caᵥ1.1^{Δe29}/ClC-1^{-/-} (solid) EDL in the presence of 20 μM verapamil. Dashed lines represent accumulated force. Note: Traces shown in FIG. 6 are replotted here with expanded timescales to better observe myotonia.

FIGS. 15A, 15B, 15C, 15D, 15E, and 15F are a set of graphs showing that Caᵥ1.1^{Δe29} exacerbates transient weakness in myotonic muscle and is alleviated by verapamil. FIG. 15A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 15B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{e29}+9-AC (n=5) mice. FIGS. 15C and 15E show normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 5 mM verapamil (FIG. 15C) or 20 mM verapamil (FIG. 15E) added to the bath. FIG. 15D shows a plot of the average peak tetanic EDL forces normalized to the initial stimulus, elicited by 44 subsequent 100 Hz, 500 ms tetanic stimulations separated by four seconds from WT+5 mM verapamil (n=5), WT+9-AC+5 mM verapamil (n=5), Caᵥ1.1^{Δe29}+5 mM verapamil (n=5) and Caᵥ1.1^{Δe29}+9-AC+5 mM verapamil (n=5) EDLs. FIG. 15F shows a plot of the average peak tetanic EDL forces normalized to the initial stimulus, elicited by 44 subsequent 100 Hz, 500 ms tetanic stimulations separated by four seconds from WT+20 mM verapamil (n=5), WT+9-AC+20 mM verapamil (n=5), Caᵥ1.1^{Δe29}+20 mM verapamil (n=5) and Caᵥ1.1^{Δe29}+9-AC+20 mM verapamil (n=5) EDLs. Dashed lines in FIGS. 15D and 15F represent average data presented in FIG. 15B as a reference for pre-treatment. Symbols, closed circles, mean±SEM. Note: Contralateral EDLs were used when possible.

FIGS. 16A and 16B are a set of graphs showing that transient weakness is absent in WT and Caᵥ1.1^{Δe29} muscle. FIG. 16 shows representative traces of the first 15, tetani (150 Hz, 500 ms) separated by 4 seconds in 20-wk WT and Caᵥ1.1^{Δe29} EDL muscle. FIG. 16B shows a plot of the average peak tetanic EDL forces normalized to the initial stimulus, elicited by 44 subsequent 100 Hz, 500 ms tetanic stimulations separated by four seconds from EDLs isolated from 20-wk WT and Caᵥ1.1^{Δe29} mice. Symbols, closed circles, mean±SEM.

FIGS. 17A, 17B, 17C, 17D, 17E, and 17F are a set of graphs showing that Caᵥ1.1^{Δe29} significantly exacerbates myotonia. FIGS. 17A, 17B, and 17C (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 17A, left) and presence of 5 mM (FIG. 17B, left) and 20 mM (FIG. 17C, left) verapamil added to the bath (pre-treatment). Dashed lines represent accumulated force production.

FIGS. 17A, 17B, and 17C (right) show a plot of average integration normalized to specific force depicted in respective left panels. FIGS. 17D, 17E, and 17F (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 17D, left) and presence of 5 mM (FIG. 17E, left) and 20 mM (FIG. 17F, left) verapamil added to the bath (pre-treatment). Dashed lines represent accumulated force production. FIGS. 17D, 17E, and 17F (right) show a plot of average integration normalized to specific force depicted in respective left panels. Symbols, open circles, individual mice; bars, mean and SEM. Note: Contralateral EDLs were used when possible.

FIGS. 18A, 18B, 18C, 18D, 18E, and 18F are a set of graphs showing that verapamil treatment does not reduce peak contraction force of non-myotonic and myotonic WT and Caᵥ1.1^{Δe29} mouse muscle. Representative specific force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 18A, left) and presence of 5 mM (FIG. 18B, left) and 20 mM (FIG. 18C, left) verapamil added to the bath (pre-treatment). Dashed lines represent accumulated force production. FIGS. 18A, 18B, and 18C (right) show a plot of average integration of specific force depicted in respective left panels. FIGS. 18D, 18E, and 18F (left) show representative specific force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 18D, left) and presence of 5 mM (FIG. 18E, left) and 20 mM (FIG. 18F, left) verapamil added to the bath (pre-treatment). Dashed lines represent accumulated force production. FIGS. 18D, 18E, and 18F (right) show a plot of average integration of specific force depicted in respective left panels. Symbols, open circles, individual mice; bars, mean and SEM. Note: Contralateral EDLs were used when possible.

FIGS. 19A and 19B are a set of graphs showing that Ca_{V}1.1^{De29} and CC-1^{-/-} alleles exhibit synthetic lethality, and result in significantly reduced body weight and severe muscle weakness in mice. Amlodipine treatment significantly improved survival and percent body weight change. FIG. 19A shows the results of Kaplan-Meier survival analysis of WT (n=10), Caᵥ1.1^{Δe29/+} (n=13), Caᵥ1.1^{Δe29} (n=15), ClC-1^{-/-} (n=40), Caᵥ1.1^{Δe29/+}/ClC-1^{-/-} (n=16), Caᵥ1.1^{Δe29/Δe29}/ClC-1^{-/-} (n=21), and Caᵥ1.1^{Δe29}/ClC-1^{-/-}+5 mg/kg/day amlodipine (n=6). FIG. 19B show percent body weight change from weaning at 10 weeks. Symbols, open circles, individual mice; bars and closed circles, means±SEM.

FIGS. 20A and 20B are a set of graphs showing that amlodipine treatment significantly improves the time of righting reflex in Caᵥ1.1^{Δe29}/ClC-1^{-/-} mice compared to untreated. FIG. 20A shows weekly time of righting reflex analysis of Caᵥ1.1^{Δe29}/ClC-1^{-/-}, ClC-1^{-/-}, Caᵥ1.1^{Δe29}, WT, Caᵥ1.1^{Δe29}/ClC-1^{-/-}+amlodipine (purple) mice. FIG. 20B shows average time of righting reflex in vehicle and amlodipine treated mice at 10- and 20-week of age. Note: Untreated Caᵥ1.1^{Δe29}/ClC-1^{-/-} mice do not survive to 20-week of age, therefore the last recording before death is documented. Box notes Q1, median, and Q3; whiskers show minimum and maximum. Symbols, closed circles, individual mice; open circles, means±SEM.

FIGS. 21A, 21B, and 21C are a set of graphs showing Caᵥ1.1^{Δe29}/ClC-1^{-/-} mice have altered respiration compared to WT, Caᵥ1.1^{Δe29}/ClC-1^{-/-} mice treated with amlodipine do not have a significantly different frequency or minute ventilation compared to WT. Whole-body plethysmography for 10-wk (left) and 20-wk (right) old mice in the indicated genotype and treatment groups. Frequency of respiration (breaths/min) (FIG. 21A), tidal volume of respiration (mL) (FIG. 21B), and minute ventilation (FIG. 21C) are shown. Symbols, open circles, individual mice; bars and closed circles, means±SEM. Note: Untreated Caᵥ1.1^{Δe29}/ClC-1^{-/-} mice do not survive to 20-week of age

FIGS. 22A and 22B are a set of graphs showing that EDL isolated from Caᵥ1.1^{Δe29}/ClC-1^{-/-} mice treated with amlodipine have similar muscle function to untreated ClC-1^{-/-} alone mice. FIG. 22A shows representative specific force traces elicited 150 Hz (500 ms) tetanic (right) stimulation of EDL muscle isolated from 20-wk mice. FIG. 22B shows average tetanic force generated during a 500 ms, 150 Hz tetanic stimulation in EDL muscle isolated from 20-week mice. FIG. 22C shows a plot of average stimulation frequency dependence of specific force generation from isolated EDL muscles at 20-week of age in the indicated genotype and treatment groups. Symbols, closed circles, means±SEM.

FIGS. 23A, 23B, and 23C are a set of graphs showing that amlodipine treatment improves muscle function in diaphragm strip isolated from Caᵥ1.1^{Δe29}/ClC-1^{-/-} mice treated with amlodipine has a force generation reduction compared to WT diaphragm. FIG. 23A shows representative specific force traces elicited 150 Hz (500 ms) tetanic (right) stimulation of diaphragm strip isolated from 20-wk mice. FIG. 23B shows average tetanic force generated during a 500 ms, 150 Hz tetanic stimulation in diaphragm strip isolated from 20-week mice. FIG. 23C shows a plot of average stimulation frequency dependence of specific force generation from isolated diaphragm strip at 20-week of age in the indicated genotype and treatment groups. Symbols, closed circles, means SEM.

FIGS. 24A, 24B, 24C, and 24D are a set of graphs showing that Caᵥ1.1^{Δe29} exacerbates transient weakness in myotonic muscle and is alleviated by amlodipine. FIG. 24A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 24B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. FIG. 24C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM amlodipine (FIG. 24C) added to the bath. FIG. 24D shows a plot of the average peak tetanic EDL forces normalized to the initial stimulus, elicited by 44 subsequent 100 Hz, 500 ms tetanic stimulations separated by four seconds from WT+10 mM amlodipine (n=5), WT+9-AC+10 mM amlodipine (n=5), Caᵥ1.1^{Δe29}+10 mM amlodipine (n=5) and Caᵥ1.1^{Δe29}+9-AC+10 mM amlodipine e (n=5) EDLs. Dashed lines in FIG. 24D represent average data presented in FIG. 24B as a reference for pre-treatment. Symbols, closed circles, mean±SEM. Note: Contralateral EDLs were used when possible.

FIGS. 25A, 25B, 25C, and 25D are a set of graphs showing that Caᵥ1.1^{Δe29} exacerbates transient weakness in myotonic muscle and is alleviated by diltiazem. FIG. 25A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 25B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. FIG. 25C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM diltiazem (FIG. 25C) added to the bath. FIG. 25D shows a plot of the average peak tetanic EDL forces normalized to the initial stimulus, elicited by 44 subsequent 100 Hz, 500 ms tetanic stimulations separated by four seconds from WT+10 mM diltiazem (n=5), WT+9-AC+10 mM diltiazem (n=5), Caᵥ1.1^{Δe29}+10 mM diltiazem (n=5) and Caᵥ1.1^{Δe29}+9-AC+10 mM diltiazem e (n=5) EDLs. Dashed lines in (FIG. 25D) represent average data presented in (FIG. 25B) as a reference for pre-treatment. Symbols, closed circles, mean±SEM. Note: Contralateral EDLs were used when possible.

FIGS. 26A, 26B, 26C, and 26D are a set of graphs showing that Caᵥ1.1^{Δe29} exacerbates transient weakness in myotonic muscle and is alleviated by nifedipine. FIG. 26A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 26B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. FIG. 26C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM nifedipine (FIG. 26C) added to the bath. FIG. 26D shows a plot of the average peak tetanic EDL forces normalized to the initial stimulus, elicited by 44 subsequent 100 Hz, 500 ms tetanic stimulations separated by four seconds from WT+10 mM nifedipine (n=5), WT+9-AC+10 mM nifedipine (n=5), Caᵥ1.1^{Δe29}+10 mM nifedipine (n=5) and Caᵥ1.1^{Δe29}+9-AC+10 mM nifedipine e (n=5) EDLs. Dashed lines in FIG. 26D represent average data presented in FIG. 26B as a reference for pre-treatment. Symbols, closed circles, mean±SEM. Note: Contralateral EDLs were used when possible.

FIGS. 27A and 27B are a set of graphs showing that transient weakness is absent in WT and Caᵥ1.1^{Δe29} muscle. FIG. 27A shows representative traces of the first 15, tetani (150 Hz, 500 ms) separated by 4 seconds in 20-wk WT and Caᵥ1.1^{Δe29} EDL muscle. FIG. 27B shows a plot of the average peak tetanic EDL forces normalized to the initial stimulus, elicited by 44 subsequent 100 Hz, 500 ms tetanic stimulations separated by four seconds from EDLs isolated from 20-wk WT and Caᵥ1.1^{Δe29} mice. Symbols, closed circles, mean±SEM.

FIGS. 28A, 28B, 28C, and 28D are a set of graphs showing that Caᵥ1.1^{Δe29} significantly exacerbates myotonia, amlodipine reduced myotonia in myotonic WT and Caᵥ1.1^{Δe29} EDL. FIGS. 28A and 28B (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 28A, left) and presence of 10 mM amlodipine (FIG. 28B, left) added to the bath. Dashed lines represent accumulated force production. FIGS. 28A and 28B (right) show a plot of average integration normalized to specific force depicted in respective left panels. FIGS. 28C and 28D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 28C, left) and presence of 10 mM amlodipine (FIG. 28D, left) added to the bath. Dashed lines represent accumulated force production. FIGS. 28C and 28D (right) show a plot of average integration normalized to specific force depicted in respective left panels. Symbols, open circles, individual mice; bars, mean and SEM. Note: Contralateral EDLs were used when possible.

FIGS. 29A, 29B, 29C, and 29D are a set of graphs showing that Caᵥ1.1^{Δe29} significantly exacerbates myotonia, diltiazem reduced myotonia in myotonic WT, and Caᵥ1.1^{Δe29} EDL. FIGS. 29A and 29B (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 29A, left) and presence of 10 mM diltiazem (FIG. 29B, left) added to the bath. Dashed lines represent accumulated force production. FIGS. 29A and 29B (right) show a plot of average integration normalized to specific force depicted in respective left panels. FIGS. 29C and 29D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 29C, left) and presence of 10 mM diltiazem (FIG. 29D, left) added to the bath. Dashed lines represent accumulated force production. FIGS. 29C and 29D, right show a plot of average integration normalized to specific force depicted in respective left panels. Symbols, open circles, individual mice; bars, mean, and SEM. Note: Contralateral EDLs were used when possible.

FIGS. 30A, 30B, 30C, and 30D are a set of graphs showing that Caᵥ1.1^{Δe29} significantly exacerbates myotonia, nifedipine reduced myotonia in myotonic WT and Caᵥ1.1^{Δe29} EDL. FIGS. 30A and 30B, left) Normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 30A, left) and presence of 75 nM nifedipine (FIG. 30B, left) added to the bath. Dashed lines represent accumulated force production. FIGS. 30A and 30B (right) show a plot of average integration normalized to specific force depicted in respective left panels. FIGS. 30C and 30D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 30C, left) and presence of 75 nM nifedipine (FIG. 30D, left) added to the bath. Dashed lines represent accumulated force production. FIGS. 30C and 30D (right) show a plot of average integration normalized to specific force depicted in respective left panels. Symbols, open circles, individual mice; bars, mean and SEM. Note: Contralateral EDLs were used when possible.

FIGS. 31A, 31B, and 31C are a set of graphs showing that the combination of HSA^{LR} with Mbnl1^{-/-} exhibit synthetic lethality, and result in significantly reduced survival and body weight, and prolonged time of righting. FIG. 31A shows the results of Kaplan-Meier survival analysis of HSA^{LR}/Mbnl1^{+/+}+vehicle, HSA^{LR}/Mbnl1^{+/+}+100 mg/kg/day verapamil, HSA^{LR}/Mbnl1^{-/-}+vehicle, and HSA^{LR}/Mbnl1^{-/-}+100 mg/kg/day verapamil. FIG. 31B shows average week-to-week body weight change. FIG. 31C shows weekly time of righting reflex analysis of HSA^{LR}/Mbnl1^{+/+}+vehicle, HSA^{LR}/Mbnl1^{+/+}+100 mg/kg/day verapamil, HSA^{LR}/Mbnl^{-/-}+vehicle, and HSA^{LR}/Mbnl1^{-/-}+100 mg/kg/day verapamil mice. Symbols, closed circles and error, means±SEM.

FIGS. 32A, 32B, and 32C are a set of graphs showing that the combination of HSA^{LR} with Mbnl1^{-/-} exhibit synthetic lethality, and result in significantly reduced survival and body weight, and prolonged time of righting. Amlodipine treatment significantly improved survival and time of righting. FIG. 32A shows the results of Kaplan-Meier survival analysis of HSA^{LR}/Mbnl1^{+/+}+vehicle, HSA^{LR}/Mbnl1^{+/+}+100 mg/kg/day amlodipine, HSA^{LR}/Mbnl1^{-/-}+vehicle, and HSA^{LR}/Mbnl1^{-/-}+100 mg/kg/day amlodipine. Untreated HSA^{LR}/Mbnl1^{-/-} mice have severely reduced lifespans. FIG. 32B shows average week-to-week body weight change. FIG. 32B shows weekly time of righting reflex analysis of HSA^{LR}/Mbnl1^{+/+}+vehicle, HSA^{LR}/Mbnl1^{+/+}+100 mg/kg/day amlodipine, HSA^{LR}/Mbn^{-/-}+vehicle, and HSA^{LR}/Mbnl1^{-/-}+100 mg/kg/day amlodipine mice. Statistical Analysis: One-way or Two-way ANOVA with multiple comparisons were used to test for significant differences between each of the groups tested. For survival, each group was compared to each other group Kaplan-Meier log-rank test. For all figures asterisks note p-values as follows, *P<0.05, **P<0.01, ***P<0.001, and ****P<0.0001 was used.

### DETAILED DESCRIPTION OF THE INVENTION

This disclosure relates to the unexpected discovery that agents capable of inhibiting calcium channels, such as calcium channel blockers, are effective in treating or alleviating the symptoms of myotonic disorders. In patients who have been diagnosed with the condition, the methods of this disclosure can, e.g., reverse or inhibit the worsening of symptoms related to such condition and/or prevent development of new symptoms.

### Methods for Treating Myotonic Disorders

Accordingly, this disclosure provides methods of treating a myotonic disorder in a subject in need thereof. In some embodiments, the methods of the disclosure use a calcium channel blocker to reverse or prevent symptoms of a myotonic disorder (e.g., muscular dystrophy, including myotonic dystrophy type 1 (DM1) or myotonic dystrophy type 2 (DM2)) by inhibiting transient paralysis, a state of paralysis following myotonia, caused by Cav1.1 activity, or/and by reducing short-term and/or long-term harms of myotonia-associated calcium entry on muscle structure or force generation capacity. In some embodiments, the methods are effective in treating a muscular dystrophy patient by reducing muscle pain, stiffness, weakness, or delayed relaxation, and protecting against long-term muscle deterioration.

As used herein, the term “myotonia” refers to any disorder or condition characterized by tonic spasm or temporary rigidity of a muscle and, in particular, the decreased relaxation of a muscle following a sustained contraction. Examples of disorders that exhibit myotonia include myotonic dystrophy, myotonia congenital, and paramyotonia congenital.

As used herein, the term “dystrophy” refers to any disorder or condition, particularly genetic conditions, characterized by degeneration in tissues, such as muscular tissue. In some embodiments, examples of muscular dystrophy include Duchenne Muscular Dystrophy, Becker Muscular Dystrophy, Emery-Dreifus Muscular Dystrophy, Facioscapulohumeral Muscular Dystrophy, Limb-Girdle Muscular Dystrophy, Myotonic Muscular Dystrophy, and Oculopharyngeal Muscular Dystrophy.

As used herein, the term “muscular dystrophy,” refers to any hereditary condition or disorder that affect skeletal muscle and is characterized by progressive muscle weakness, defects in muscle proteins, and, ultimately, muscle cell death. This term broadly encompasses any condition that involves at least one and typically more symptoms, including muscle pain, muscle weakness, muscle stiffness, difficulty in walking, myotonia, fatigue, scoliosis, axonal peripheral neuropathy, cardiomyopathy, cardiac arrhythmia, mental retardation, hypersomnia, sleep apnea, iridescent posterior subcapsular cataracts, insulin insensitivity, type II diabetes mellitus, premature balding, testicular failure, infantile hypotonia, and respiratory deficits. Not all symptoms need to be present. For example, a person with muscular dystrophy may exhibit cataracts or mild myotonia, but not muscular weakness or cardiac arrhythmia. Similarly, a person with muscular dystrophy may exhibit muscular pain and weakness but not hypersomnia or sleep apnea.

As used herein, the terms “myotonic dystrophy type 1” or “DM1” is also known as Steinert's Disease, a condition where the patient has an abnormally large number of CTG repeats in the patient's DMPK gene. Typically, symptoms are noted in individuals with 50 or greater CTG repeats. DM1 includes patients with mild, classic, and congenital forms (phenotypes) of the disease.

As used herein, the terms “myotonic dystrophy type 2,” “DM2,” “proximal myotonic myopathy,” and “PROMM” refer to a condition where the patient has an abnormally large number of CCTG repeats in their CNBP (ZNF9) gene. General symptoms are noted in individuals that have at least 75 CCTG repeats.

Typically, the number and severity of muscular dystrophy symptoms depend on the type and severity of the genetic defect. For example, with DM1, as the CTG copy number increases, the age at which symptoms manifest decreases, with patients with higher copy numbers having more severe symptoms at a particular age compared to patients with a lower copy number. For milder forms of the disease, a patient may initially be asymptomatic and present with physical manifestations of the disease once adolescence or adulthood is reached.

In some embodiments, the myotonic disorder is myotonic dystrophy, autosomal dominant or recessive myotonia congenita, or sodium channel myotonia. In some embodiments, myotonic dystrophy is myotonic dystrophy type 1 (DM1) or myotonic dystrophy type 2 (DM2). In some embodiments, myotonic dystrophy is associated with abnormal alternative splicing of RNA encoding CIC-1 chloride channel or Cav1.1 calcium channel. Cav1.1, also known as the calcium channel, voltage-dependent, L type, alpha 1S subunit (CACNA1S; Uniprot accession code: Q13698), is a protein which in humans is encoded by the CACNA1S gene.

In some embodiments, the myotonia congenita is Becker or Thomsen myotonia congenita. In some embodiments, the sodium channel myotonia is paramyotonia congentia, hyperkalemic periodic paralysis, or potassium-aggravated myotonia. In some embodiments, the subject has one or more mutations in SCN4A, Clcn1, or Cac1ns, or has alternative splice isoforms of Clcn1 or Cac1ns.

In some embodiments, the method comprises administering to the subject a therapeutically effective amount of an agent (e.g., a calcium channel blocker) that inhibits a calcium channel (e.g., Cav1.1 calcium channel) in a cell of the subject. In some embodiments, the agent comprises a calcium channel blocker, a stereoisomer thereof, a derivative/analog thereof, a prodrug thereof, a metabolite thereof, or a pharmaceutically acceptable salt thereof, or a combination thereof.

As used herein, the term “calcium channel blocker,” “channel antagonist,” “calcium channel inhibitor,” or “calcium entry blocker” refers to an agent that blocks voltage-dependent calcium channels. Exemplary calcium channel blockers include, but are not limited to, amlodipine, felodipine, isradipine, lacidipine, nicardipine, nifedipine, niguldipine, niludipine, nimodipine, nisoldipine, nitrendipine, nivaldipine, ryosidine, anipamil, diltiazem, fendiline, flunarizine, gallopamil, mibefradil, prenylamine, tiapamil, verapamil, perhexiline maleate, fendiline, prenylamine, a stereoisomer, a derivative thereof or a pharmaceutically acceptable salt thereof, and a combination thereof.

In some embodiments, the agent is selected from verapamil, amlodipine, diltiazem, nifedipine, a stereoisomer, a derivative thereof or a pharmaceutically acceptable salt thereof, and a combination thereof.

Verapamil has an TUPAC name of (RS)-2-(3,4-Dimethoxyphenyl)-5-{[2-(3,4-dimethoxyphenyl)ethyl]-(methyl)amino}-2-prop-2-ylpentanenitrile, and the following chemical formula:

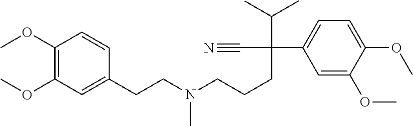

Amlodipine has an IUPAC name of (RS)-3-ethyl 5-methyl 2-[(2-aminoethoxy)methyl]-4-(2-chlorophenyl)-6-methyl-1,4-dihydropyridine-3,5-dicarboxylate, and the following chemical formula:

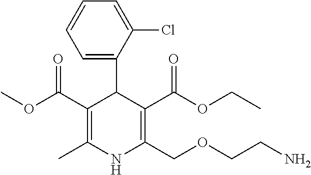 [SMILES: CCOC(=O)C1=C(COCCN)NC(C)=C(C(=O)OC)C1c1ccccc1Cl]

Diltiazem has an IUPAC name of cis-(+)-[2-(2-Dimethylaminoethyl)-5-(4-methoxyphenyl)-3-oxo-6-thia-2-azabicyclo[5.4.0]undeca-7,9,11-trien-4-yl]ethanoate, and the following chemical formula:

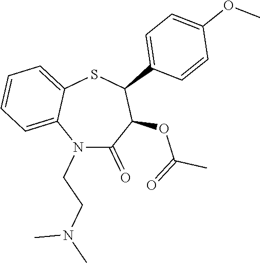 [SMILES: COc1ccc([C@@H]2Sc3ccccc3N(CCN(C)C)C(=O)[C@@H]2OC(C)=O)cc1]

Nifedipine has an IUPAC name of 3,5-dimethyl 2,6-dimethyl-4-(2-nitrophenyl)-1,4-dihydropyridine-3,5-dicarboxylate, and the following chemical formula:

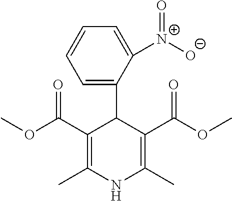

As used herein, the terms “treating,” “treat,” and “treatment” include (i) preventing a disease, pathologic or medical condition from occurring (e.g., prophylaxis); (ii) inhibiting the disease, pathologic or medical condition or arresting its development; (iii) relieving or ameliorating the disease, pathologic or medical condition; and/or (iv) diminishing symptoms associated with the disease, pathologic or medical condition. Thus, the terms “treat,” “treatment,” and “treating” can extend to prophylaxis and can include preventing, prevention, lowering, stopping, or reversing the progression or severity of the condition or symptoms being treated. As such, the term “treatment” can include medical, therapeutic, and/or prophylactic administration, as appropriate. The term “treating” or “treatment” thus can include reversing, reducing, or arresting the symptoms, clinical signs, and underlying pathology of a condition in a manner to improve or stabilize a subject's condition.

The term “ameliorate,” as used herein, refers to the effects of administering an agent to a patient (e.g., a myotonic dystrophy patent) that result in any indicia of success in the prevention, reduction, or reversal of one or more symptoms related to the condition. Reduction may be indicated in lesser severity, delayed onset of symptoms, or a slowing of disease progression. The prevention, reduction, or reversal of symptoms can be measured based on objective parameters, such as the results of a physical examination or laboratory test (i.e., blood test), decreased need for medication, decreased need for supportive measures (i.e., use of a ventilator), or increase in mobility. The prevention, reduction, or reversal of symptoms can also be measured based on subjective parameters, such as a reduction in pain or stiffness or an increase in a patient's mobility and sense of wellbeing.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

In some embodiments, the agent is administered to the subject intratumorally, intravenously, subcutaneously, intraosseously, orally, transdermally, sublingually, in sustained release, in controlled release, in delayed release, or as a suppository.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. A “therapeutically effective amount” of a compound with respect to the subject method of treatment refers to an amount of the compound in a preparation which, when administered as part of a desired dosage regimen (to a mammal, e.g., a human) alleviates a symptom, ameliorates a condition, or slows the onset of disease conditions according to clinically acceptable standards for the disorder or condition to be treated, e.g., at a reasonable benefit/risk ratio applicable to any medical treatment.

The compound can be conveniently administered in a unit dosage form, for example, containing 5 to 1000 mg/m², conveniently 10 to 750 mg/m², most conveniently, 50 to 500 mg/m² of active ingredient per unit dosage form. In some embodiments, a dose may be presented in a single dose or as divided doses administered at appropriate intervals, for example, as two, three, four or more sub-doses per day. The sub-dose itself may be further divided, e.g., into a number of discrete, loosely spaced administrations.

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In some embodiments, the method comprises administering to the subject one or more doses of the agent comprising from 0.1 mg/kg/body weight to about 1000 mg/kg/body weight or more, e.g., about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more, or any range derivable therein.

In some embodiments, the agent is administered to the subject at one or more doses of from about 0.01 to about 100 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, or about 99 mg/kg) of body weight of the subject. In some embodiments, the agent is administered at one or more doses of from about 1 to about 50 mg/kg of body weight of the subject. In some embodiments, the agent is administered at one or more doses of from about 8 to about 16 mg/kg of body weight of the subject.

In some embodiments, one or more doses of the agent are administered at least every 1 day, every 2 days, every 3 days, every 4 days, every 5 days, every 6 days, every 7 days, every 8 days, every 9 days, every 10 days, every 11 days, every 12 days, every 13 days, every 14 days, every 15 days, every 16 days, every 17 days, every 18 days, every 19 days, every 20 days, every 21 days, every 22 days, every 23 days, every 24 days, every 25 days, every 26 days, every 27 days, every 28 days, every 29 days, every 30 days, every 31 days, every 32 days, every 33 days, every 34 days, every 35 days, every 36 days, every 37 days, every 38 days, every 39 days, every 40 days, every 41 days, every 42 days, every 43 days, every 44 days, every 45 days, every 46 days, every 47 days, every 48 days, every 49 days, every 50 days, every 51 days, every 52 days, every 53 days, every 54 days, every 55 days, every 56 days, every 57 days, every 58 days, every 59 days, every 60 days, every 61 days, every 62 days, every 63 days, every 64 days, every 65 days, every 66 days, every 67 days, every 68 days, every 69 days, every 70 days, every 71 days, every 72 days, every 73 days, every 74 days, every 75 days, every 76 days, every 77 days, every 78 days, every 79 days, every 80 days, every 81 days, every 82 days, every 83 days, every 84 days, every 85 days, every 86 days, every 87 days, every 88 days, every 89 days, every 90 days, every 91 days, every 92 days, every 93 days, every 94 days, every 95 days, every 96 days, every 97 days, every 98 days, every 99 days, every 100 days, every 101 days, every 102 days, every 103 days, every 104 days, every 105 days, every 106 days, every 107 days, every 108 days, every 109 days, every 110 days, every 111 days, every 112 days, every 113 days, every 114 days, every 115 days, every 116 days, every 117 days, every 118 days, every 119 days, or every 120 days.

In some embodiments, the treatment produces a therapeutic effect selected from reduced myotonia, reduced body weight loss, improved survival, improved muscle function, improved time of righting reflex, improved respiratory function, and improved diaphragm strength. In some embodiments, the treatment results in at least 50% improvement in myotonia, body weight loss, survival, muscle function, time of righting reflex, respiratory function, or diaphragm strength as compared to an untreated subject.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

In another aspect, this disclosure also provides use of an agent that inhibits a calcium channel in the manufacture of a medicament for treating a myotonic disorder in a subject in need thereof. In some embodiments, the myotonic disorder is myotonic dystrophy, autosomal dominant or recessive myotonia congenita, or sodium channel myotonia. In some embodiments, the calcium channel is a Cav1.1 calcium channel.

In some embodiments, the agent is selected from verapamil, amlodipine, diltiazem, nifedipine, a stereoisomer thereof, a derivative/analog thereof, a prodrug thereof, a metabolite thereof, or a pharmaceutically acceptable salt thereof, or a combination thereof.

### Pharmaceutical Compositions and Kits

Also provided in this disclosure is a pharmaceutical composition comprising (i) an agent (e.g., calcium channel blocker) described above, a stereoisomer thereof, a derivative/analog thereof, a prodrug thereof, a metabolite thereof, or a pharmaceutically acceptable salt thereof, and (ii) optionally a pharmaceutically acceptable carrier.

In some embodiments, pharmaceutical compositions described herein, e.g., for treating a myotonic disorder in a subject in need thereof, comprise: a calcium channel blocker selected from verapamil, amlodipine, diltiazem, nifedipine, a stereoisomer thereof, a derivative/analog thereof, a prodrug thereof, a metabolite thereof, or a pharmaceutically acceptable salt thereof, and a combination thereof.

In some embodiments, pharmaceutical compositions may comprise, for example, at least about 0.1% of a compound. In other embodiments, an active compound may comprise between about 2% to about 75% of the weight of the unit, or between about 25% to about 60%, for example, and any range derivable therein.

In some embodiments, the pharmaceutical compositions further comprise one or more agents selected from ranolazine mexiletine, flecainide, tocainide, phenytoin, carbamazepine, lamotrigine, a stereoisomer thereof, a derivative/analog thereof, a prodrug thereof, a metabolite thereof, or a pharmaceutically acceptable salt thereof, and a combination thereof.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

In some embodiments, the agents used herein (e.g., calcium channel blockers) may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “St,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by a structure without indicating the stereochemistry, and the compound has at least one chiral center, it is to be understood that the name or structure encompasses one enantiomer of compound free from the corresponding optical isomer, a racemic mixture of the compound and mixtures enriched in one enantiomer relative to its corresponding optical isomer.

When a disclosed compound is named or depicted by a structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing directly or indirectly, the activity disclosed herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The compounds can form salts which are also within the scope of this disclosure. Unless otherwise indicated, reference to an inventive compound is understood to include reference to one or more salts thereof. The term “salt(s)” denotes acidic and/or basic salts formed with inorganic and/or organic acids and bases. In addition, the term “salt(s) may include zwitterions (inner salts), e.g., when a compound contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the disclosure. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long-chain halides (e.g., decyl, lauryl, myristyl, and stearyl chlorides, bromides and iodides), aralkyl halides (e.g., benzyl and phenethyl bromides), and others. In some embodiments, examples of salts include monohydrochloride, hydrogensulfate, methanesulfonate, phosphate or nitrate salts.

Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pgs 113-191 (Harwood Academic Publishers, 1991); and (d) Hydrolysis in Drug and Prodrug Metabolism, Bernard Testa and Joachim M. Mayer, (Wiley-VCH, 2003).

In addition, the compounds, subsequent to their preparation, can be isolated and purified to obtain a composition containing an amount by weight equal to or greater than 99% of a compound (“substantially pure”), which is then used or formulated as described herein. Such “substantially pure” compounds are also contemplated herein as part of the present disclosure.

“Stable compound” and “stable structure” are meant to indicate a compound that is sufficiently robust to survive isolation to a useful degree of purity from a reaction mixture, and formulation into an efficacious therapeutic agent. The present disclosure is intended to embody stable compounds.

The compounds of the present disclosure are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

Compounds and/or pharmaceutically acceptable salts thereof can be administered by any means suitable for the condition to be treated, which can depend on the need for site-specific treatment or quantity of the compound to be delivered. Also embraced within this disclosure is a class of pharmaceutical compositions comprising a compound and/or pharmaceutically acceptable salts thereof, and one or more non-toxic, pharmaceutically-acceptable carriers and/or diluents and/or adjuvants (collectively referred to herein as “carrier” materials) and, if desired, other active ingredients. The compounds may be administered by any suitable route, e.g., in the form of a pharmaceutical composition adapted to such a route, and in a dose effective for the treatment intended. The compounds and compositions of the present disclosure may, for example, be administered orally, mucosally, rectally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate, and a disintegrating agent such as crospovidone. The carrier mixture may be filled into a gelatin capsule or compressed as a tablet. The pharmaceutical composition may be administered as an oral dosage form or an infusion, for example.

Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, e.g., in physiologically compatible buffers such as Hank's solution or Ringer's solution. In addition, the agents may be formulated in solid form and redissolved or suspended immediately prior to use. Lyophilized forms are also included.

For oral administration, the pharmaceutical composition may be in the form of, for example, a tablet, capsule, liquid capsule, suspension, or liquid. The pharmaceutical composition is e.g., made in the form of a dosage unit containing a particular amount of the active ingredient. For example, the pharmaceutical composition may be provided as a tablet or capsule comprising an amount of active ingredient in the range of from about 0.1 to 1000 mg, e.g., from about 0.25 to 250 mg, and e.g., from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium croscarmellose, corn starch, and alginic acid; binding agents, such as, for example, starch, gelatin, polyvinylpyrrolidone, and acacia; and lubricating agents, such as, for example, magnesium stearate, stearic acid, and talc. Additionally, a tablet can either be uncoated or coated by known techniques to either mask the bad taste of an unpleasant tasting drug or delay disintegration and absorption of the active ingredient in the gastrointestinal tract, thereby sustaining the effects of the active ingredient for a longer period. Exemplary water-soluble taste-masking materials include, but are not limited to, hydroxypropyl-methylcellulose and hydroxypropyl-cellulose. Exemplary time delay materials include, but are not limited to, ethylcellulose and cellulose acetate butyrate.

Hard gelatin capsules can, for example, be prepared by mixing at least one compound and/or at least one salt thereof with at least one inert solid diluent, such as, for example, calcium carbonate; calcium phosphate; and kaolin.

Soft gelatin capsules can, for example, be prepared by mixing at least one compound and/or at least one pharmaceutically acceptable salt thereof with at least one water-soluble carrier, such as, for example, polyethylene glycol; and at least one oil medium, such as, for example, peanut oil, liquid paraffin, and olive oil.

An aqueous suspension can be prepared, for example, by admixing at least one compound and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethylcellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long-chain aliphatic alcohols, such as, for example, heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound and/or at least one pharmaceutically acceptable salt thereof in either vegetable oil, such as, for example, Arachis oil; olive oil; sesame oil; and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisole, and alpha-tocopherol.

Dispersible powders and granules can, for example, be prepared by admixing at least one compound and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound and/or at least one pharmaceutically acceptable salt thereof can, for example, be prepared as an oil-in-water emulsion. The oily phase of the emulsions comprising compounds may be constituted from known ingredients in a known manner. The oil phase can be provided by, but is not limited to, for example, a vegetable oil, such as, for example, olive oil, Arachis oil, mineral oil, such as, for example, liquid paraffin, and mixtures thereof. While the phase may comprise merely an emulsifier, it may comprise a mixture of at least one emulsifier with a fat or an oil or with both fat and oil. Suitable emulsifying agents include, but are not limited to, for example, naturally-occurring phosphatides, e.g., soybean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. In some embodiments, a hydrophilic emulsifier is included together with a lipophilic emulsifier, which acts as a stabilizer. It is also preferred to include both oil and fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceryl distearate alone or with a wax, or other materials well known in the art.

A pharmaceutical composition described herein can also be incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier does not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid, and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

The compounds and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, or water, or with cyclodextrin (i.e., Captisol), cosolvent solubilization (i.e., propylene glycol) or micellar solubilization (i.e., Tween 80).

The sterile injectable preparation may also be a sterile injectable solution or suspension in a non-toxic, parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by (1) dissolving at least one compound in an oily phase, such as, for example, a mixture of soybean oil and lecithin; (2) combining containing oil phase with a water and glycerol mixture; and (3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butanediol; and a sterile oleaginous suspension can be prepared with a sterile, non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as alpha-tocopherol polyethylene glycol 1000 succinate, surfactants used in pharmaceutical dosage forms such as Tweens, polyethoxylated castor oil, such as cremophor surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as additives, preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt. % to 60 wt. %, e.g., 2 wt. % to 50 wt. %, solubilizer and/or skin permeation enhancer; 2 wt. % to 50 wt. %, e.g., 2 wt. % to 20 wt. %, emulsifiers; 2 wt. % to 20 wt. % emollient; and 0.01 to 0.2 wt. % preservative, with the active agent and carrier (e.g., water) making up the remainder of the formulation. A skin permeation enhancer serves to facilitate passage of therapeutic levels of active agent to pass through a reasonably sized area of unbroken skin. Suitable enhancers are well known in the art and include, for example, lower alkanols such as methanol ethanol and 2-propanol; alkyl methyl sulfoxides such as dimethylsulfoxide (DMSO), decylmethylsulfoxide (C.sub.lO MSO) and tetradecylmethyl sulfoxide; pyrrolidones such as 2-pyrrolidone, N-methyl-2-pyrrolidone and N-(-hydroxyethyl)pyrrolidone; urea; N,N-diethyl-m-toluamide; C₂-C₆ alkane diols; miscellaneous solvents such as dimethylformamide (DMF), N,N-dimethylacetamide (DMA), and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, e.g., in the range of approximately 0.25 wt. % to 30 wt. % of the formulation, e.g., in the range of approximately 0.5 wt. % to 15 wt. % of the formulation, and e.g., in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

The amounts of compounds that are administered and the dosage regimen for treating a disease condition with the compounds and/or compositions of this disclosure depends on a variety of factors, including the age, weight, sex, medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, e.g., between about 0.0025 and about 50 mg/kg body weight and, e.g., between about 0.005 to 10 mg/kg body weight, may be appropriate. The daily dose can be administered in one to four doses per day. Other dosing schedules include one dose per week and one dose per two-day cycle.

For therapeutic purposes, the active compounds of this disclosure are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethylcellulose.

Pharmaceutical compositions of this disclosure comprise at least one compound and/or at least one pharmaceutically acceptable salt thereof, and optionally an additional agent selected from any pharmaceutically acceptable carrier, adjuvant, and vehicle. Alternate compositions of this disclosure comprise a compound described herein, or a prodrug thereof, and a pharmaceutically acceptable carrier, adjuvant, or vehicle.

The compound, the composition or the pharmaceutical composition described herein can be provided in a kit. In one embodiment, the kit includes (a) a container that contains the composition and optionally (b) informational material. The informational material can be descriptive, instructional, marketing or other material that relates to the methods described herein and/or the use of the agents for therapeutic benefit. For example, kits may include instructions for the manufacturing, for the therapeutic regimen to be used, and periods of administration. In an embodiment, the kit includes also includes an additional therapeutic agent. The kit may comprise one or more containers, each with a different reagent. For example, the kit includes a first container that contains the composition and a second container for the additional therapeutic agent.

The containers can include a unit dosage of the pharmaceutical composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device. The device can be provided pre-loaded with one or both of the agents or can be empty, but suitable for loading.

### Combination Therapies

Active ingredients described herein (e.g., verapamil, ranolazine mexiletine, flecainide, tocainide, phenytoin, carbamazepine, and lamotrigine) can also be used in combination with other active ingredients. Such combinations can be selected based on the condition to be treated, cross-reactivities of ingredients and pharmaco-properties of the combination.

In some embodiments, the method further comprises administering to the subject an additional therapeutic agent or therapy. In some embodiments, the additional therapeutic agent is selected from ranolazine mexiletine, flecainide, tocainide, phenytoin, carbamazepine, lamotrigine, and a combination thereof. In some embodiments, the additional therapeutic agent or therapy is administered to the subject before, after, or concomitantly with the agent.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

It is also possible to combine a compound of the invention with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide synergy and be synergistic, i.e., the effect achieved when the active ingredients used together are greater than the sum of the effects that result from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills, or capsules, or by different injections in separate syringes. In general, during alternation therapy, an effective dosage of each active ingredient is administered sequentially, i.e., serially, whereas, in combination therapy, effective dosages of two or more active ingredients are administered together. A synergistic effect denotes an effect that is greater than the predicted purely additive effects of the individual compounds of the combination.

Combination therapy is further described by U.S. Pat. Nos. 11,103,514, 10,702,495, 9,382,215, and 6,833,373, which include additional active agents that can be combined with the compounds described herein, and additional types of ailments and other conditions that can be treated with a compound or combination of compounds described herein.

Accordingly, it is an aspect of this invention that an active agent (e.g., verapamil, ranolazine mexiletine, flecainide, tocainide, phenytoin, carbamazepine, and lamotrigine) can be used in combination with another agent or therapy method. An active agent may precede or follow treatment of the other agent by intervals ranging from minutes to weeks. In embodiments where the other agent and expression construct are applied separately to a cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with the disclosed active.

In some embodiments, one or more agents may be administered within about 1 minute, about 5 minutes, about 10 minutes, about 20 minutes about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the disclosed active agent. In certain other embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the disclosed active. In some situations, it may be desirable to extend the time period for treatment significantly; however, where several weeks (e.g., about 1, about 2, about 3, about 4, about 6, or about 8 weeks or more) lapse between the respective administrations.

Administration of the compositions of the invention to a patient will follow general protocols for the administration of therapeutics, taking into account the toxicity, if any. It is expected that the treatment cycles would be repeated as necessary. It also is contemplated that various standard therapies or adjunct therapies, as well as surgical intervention, may be applied in combination with the described active agent. These therapies include but are not limited to chemotherapy, radiotherapy, immunotherapy, gene therapy and surgery.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, a “subject” or “subject in need thereof” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In some embodiments, the subject is a human. In another embodiment, the subject is an experimental animal or animal suitable as a disease model.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

Multiple techniques of administering a compound exist in the art including, but not limited to, intravenous, oral, aerosol, parenteral, ophthalmic, pulmonary and topical administration. “Parenteral” administration of a composition includes, e.g., subcutaneous (s.c.), intravenous (i.v.), intramuscular (i.m.), or intrasternal injection, or infusion techniques.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

“Sample,” “test sample,” and “patient sample” may be used interchangeably herein. The sample can be a sample of serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample,” as used herein, generally refer to a biological material being tested for and/or suspected of containing an analyte of interest. The sample may be any tissue sample from the subject. The sample may comprise protein from the subject.

The terms “inhibit” and “antagonize,” as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down-regulate a protein, a gene, and an mRNA stability, expression, function, and activity, e.g., antagonists.

The terms “decrease,” “reduced,” “reduction,” “decrease,” or “inhibit” are all used herein generally to mean a decrease by a statistically significant amount. However, for avoidance of doubt, “reduced,” “reduction,” “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e. increasing, decreasing, and the like.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Doses are often expressed in relation to body weight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “In some embodiments,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 4%1, 3%1, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the subsequent EXAMPLES below.

Generating Caᵥ1.1^{Δe29}/CIC-1^{-/-} Mouse Line

CRISPR/Cas9 was used to generate Caᵥ1.1^{Δe29/Δe29} mice. The sequences of exon 29 were removed by targeting 50 nucleotides into an intronic sequence on either side of the intron/exon border. This ensured that both the exon and the splice donor/acceptor sequence were removed. sgRNAs for this region were paired and completed to Cas9. Complexes were injected in C57bl6/J zygotes. Surrogate mice were implanted with embryos once the blastocyst stage was reached.

Subsequently, this line was backcrossed to C57bl6 (Jackson) >6 generations to ensure no off-target mutations. The removal of exon 29 was validated by RNA isolation of tibialis anterior and Sanger sequencing of RT-PCR products between exons 27 (5′ CCAGTCGGAACAGATGAACCAC (SEQ ID NO: 1)) and 31(5′ CCGATGACCGCGTAGATGAAGA (SEQ ID NO: 2)). To generate Caᵥ1.1^{Δe29}/ClC-1^{-/-} mice, adr-mto2J mice (Jackson Labs), which have a frameshift mutation in the Clcn1 muscle-specific chloride channel causing a recessive generalized myotonia, with our Caᵥ1.1^{Δe29/Δe29} mice. Additionally, the adr-mto2J mice were used throughout the experimentation (ClC-1^{-/-}). WT C57bl6 controls were obtained from Jackson Labs. All mice were housed, bred, cared for, and experimented on in accordance with University of Rochester Committee on Animal Resources approval.

Drugging Mice with Verapamil

WT treatment control mice and Caᵥ1.1^{Δe29}/ClC-1^{-/-} mice were treated by drugging Nutra-Gel Complete Nutrition (Bio-Serv) food cups to a final dose of 200 mg/kg/day verapamil (V4629, (±)-Verapamil hydrochloride, Sigma-Aldrich). Mice were provided food cups immediately after weaning, and food was changed daily. No other food source was given, but water was provided and changed weekly along with cages.

### Body Weight Measurements

Body weight of the mice was measured every other day following weaning to the end of terminal experimentation. For statistical analysis, two-way ANOVA was performed on the average of each week's measurements. Percent body weight change at 10 weeks was determined with the first weight measurement taken post-weaning, and the body weight reached at 10 weeks. For this data, a one-way ANOVA with multiple comparisons was performed.

### Systolic Blood Pressure and Heart Rate Measurements:

Systolic blood pressure and heart rate were measured with a non-invasive tail cuff BP-2000 Blood Pressure Analysis System (Visitech Systems). Mice were allowed to acclimate to the testing room for one hour prior to the start of each day. 10-week WT mice treated with vehicle, 100 mg/kg/day, or 200 mg/kg/day were trained for five days to acclimate to the system. This was followed by five testing days starting immediately after the final training day. Throughout training and testing, 10 preliminary measurements and 20 actual measurements were taken for each mouse.

### Time of Righting Reflex

To measure verapamil's influence on myotonia and muscle strength in Caᵥ1.1^{Δe29}/ClC-1^{-/-} mice, time of righting reflex was tracked weekly after the start of verapamil treatment. Mice were held in a supine position on a level surface and then released. Two persons began timing immediately after release, and timing was stopped once all four paws were down on the surface. This was repeated for a total of 10 timed righting responses with a minimum of five minutes in between each recording to avoid myotonic warm-up. Recordings less than a second were recorded as “1,” and righting was considered a failure if righting took more than 60 seconds. If failure was reached, no further recordings were measured for that mouse for that week. The two longest and two shortest recording for each mouse was removed, and the average of the middle 6 recordings were used for analysis. Two-way ANOVA on the week-to-week results and one-way ANOAV with multiple comparisons for the timepoint data (10-week and 20-week) were performed.

### Whole Body Plethysmography

Whole body plethysmography (Buxco® Small Animal Whole Body Plethysmography, Data Science Internation) was used to measure respiratory function at 10 weeks (all groups) and 20 weeks (excluding untreated Caᵥ1.1^{Δe29}/ClC-1^{-/-}). Mice were acclimated to the system for two days, with data being collected on day three. For all three days, single mice will be placed conscious and unrestrained in WBP chamber and subjected to a 15-minute acclimation period, followed by 10 minutes of data acquisition (Yonekawa T, et al. Science advances. 2022; 8(21)). Average data from the 10-minute acquisition period for frequency (Breath/min) and tidal volume (mL) were analyzed for statistics by one-way ANOVA with multiple comparisons.

Whole-Cell Patch-Clamp Recording of Calcium Currents from Dissociated Adult FDB Fibers

Adult mice at approximately 4 weeks postnatal were sacrificed by cervical dislocation and decapitation, as approved by the University Committee on Animal Resources at the University of Rochester. Isolated Flexor digitorum brevis (FDB) muscle fibers for electrophysiology experiments were obtained by previously described methods (Lueck et al. J Gen Physiol. 2007 January; 129(1):79-94). FDB muscles were immediately micro-dissected submerged in standard electrophysiology Ringer's solution (146 mM NaCl, 5 KCl, 2 mM CaCl₂, 1 mM MgCl₂, and 10 mM HEPES at pH 7.4). Excised muscles were then digested for 1 hour in an oscillating water bath at 37° C. in Ringer's solution supplemented with 2 mg/ml Collagenase A (Roche, Mannheim, Germany).

Following enzymatic digestion, the muscles were mechanically dissociated by trituration with fire-polished Pasteur pipettes of decreasing pore diameter to obtain isolated single FDB fibers. Fibers were then plated on 35 mm plastic cell-culture dishes for experiments. Fibers were stored at room temperature in Ringer's solution until they were utilized for experiments. All patch-clamp experiments were completed within 8-hours of the sacrifice of the mouse. To record Cav1.1 calcium currents with whole-cell patch-clamp, solutions were designed to isolate calcium currents (TTX (Cayman Chemical Company, Ann Arbor, MI) used to block sodium currents, TEA, 4-AP, and cesium used to block potassium currents, and 9-AC used to block chloride currents). External recording solution contains 145 mM TEA-methanesulfonic acid, 10 mM CaCl₂, 10 mM HEPES, 2 mM MgSO₄, 1 mM 4-aminopyridine, 0.1 mM anthracene-9-carboxylic acid, and 0.001 mM tetrodotoxin pH 7.4 with TEA-OH. Low-resistance pipettes were fashioned from thin-walled borosilicate glass with a Sutter P-97 puller (Sutter Instruments, Novato, CA) and fire-polished to a resistance <1 MΩ. Pipettes were filled with an internal solution with: 140 mM Cs-aspartate, 10 mM Cs2-EGTA, 5 mM MgCl₂, and 10 mM HEPES pH 7.4 with CsOH.

Healthy muscle fibers with clear striations were sealed on to achieve a gigaseal with negative pressure then the whole-cell configuration was achieved through the application of additional negative pressure that was maintained throughout data collection. Prior to collecting current recordings, the fibers were maintained at a resting potential of −80 mV and allowed to dialyze for greater than 15 minutes to allow for the internal solution to equilibrate within the fibers. Currents were recorded with standard voltage-step protocols with 500 ms voltage steps from −50 to −80 to 80 mV at 10 mV intervals, as described in Begollari et al. (2015). Data was collected using an Axopatch 200b amplifier (Axon Instruments, San Jose, CA) and digitized with a Digidata 1550b (Axon Instruments). A −P/4 online subtraction protocol was utilized to correct linear components of leak and capacitive currents. To limit voltage errors, whole-cell parameters were adjusted, and series resistance was compensated (90%). Recordings were sampled at 10 kHz and filtered at 2 kHz. Cell capacitance and other passive properties of the muscle fibers were either obtained via Clampex 10 (Molecular Devices) or calculated based on integration of a 10-mV voltage step from resting. Currents were normalized to fiber capacitance to obtain current densities (pA/pF). All experiments were completed at room temperature. Peak current densities obtained for the family of voltage steps were plotted and fit with the following equation to obtain I-V curves.

$$I = Gmax * ( V - V rev ) / \{ 1 + exp [ - ( V - V 1 / 2 ) / kG ] \}$$

In this equation, I correspond to the peak current of a specific test potential normalized to cell capacitance, Gmax is the maximum calcium conductance, V1/2 is the half-maximal activation potential, Vrev is the reversal potential, and kG is the slope factor.

### Histology and Fiber Typing

For hematoxylin and eosin (H and E) staining, as well as fiber typing by immunohistochemistry, tibialis anterior and diaphragm were isolated 10-week and 20-week mice (n=5/group) and snap frozen. Transverse 10-micron cryosections from each muscle were collected on glass slides. For H and E, slides will be submerged in hematoxylin for 3.5 min, followed by a 1 dip into ddH₂O, 1% HCl, ddH₂O, ammonia water, ddH₂O, and submerged in eosin for 5 min. Slides were dehydrated by two 30-second incubations in 95% ethanol, two 30-second incubations in 100% ethanol, and one 30-second incubation followed by a 5-minute incubation in Hemo-De (xylene substitute). The slides were then mounted with Permount (Fisher) and imaged on an Olympus BX53. Immunohistochemistry to determine fiber type was performed as previously reported (Bachman J F, et al. Development. 2018; 145(20) Epub 20181025). Briefly, slides were permeabilized once with PBST (PBS with 0.2% Triton X-100) for 10 minutes and then blocked for 30 minutes in 10% normal goat serum (added later) at room temperature. Next, slides were blocked for 60 minutes in 3% AffiniPure Fab Fragment anti-mouse IgG (H&L) (Jackson Immunoresearch) (0.1 mg/ml) with 2% NGS in PBS at room temperature, then washed three times for 10 minutes each in PBS. Slides were incubated for 14-18 hrs at 4° C. in primary antibodies (noted in Table B). Next, slides were washed three times for 10 minutes PBS at room temperature then secondary antibodies (see Table B) were added and incubated for 60 minutes. After another set of three washes for 10 minutes in 1×PBS at room temperature, slides were mounted with Fluromount-G (Invitrogen). Slides were imaged using a Keyance BZ-X810 All-in-One Florescence Microscope (Leica).

### In Vitro Muscle Contraction

Muscle strength and frequency dependence were measured by in vitro muscle contraction performed on an Aurora Scientific 1200A in vitro system equipped with 809B muscle testing system with a 300C-LR force transducer and 701C stimulator (Aurora Scientific). For this experiment, 10- and 20-week WT, WT+verapamil, Caᵥ1.1^{Δe29}, ClC-1^{-/-}, untreated Caᵥ1.1^{Δe29}/ClC-1^{-/-} (only at 10-weeks), and Caᵥ1.1^{Δe29}/ClC-1^{-/-}+verapamil mice were used. Mice were anesthetized by 2% inhaled isoflurane. For the EDL muscle, the proximal and distal tendons of the EDL were exposed after removal of the tibial anterior and tied using a suture thread. The proximal tendon was set on an immobile post, and the distal tendon was hooked to the force transducer. For diaphragm, a 4 mm strip was dissected from the right costal hemidiaphragm as in Hakim, C. et al. (2019). A suture was used to connect the diaphragm strip to the stationary post from the ribs, and the central tendon was secured to the force transducer (Hakim C H, et al., Sci Rep. 2019; 9(1):19453). For both muscle groups, the muscles were submerged between platinum electrodes in warmed (30° C.) and oxygenated (95% O₂ and 5% CO₂) Ringer's buffer (1.2 mM NaH₂PO₄, 1 mM MgSO₄, 4.83 mM KCl, 137 mM NaCl, 2 mM CaCl₂, 10 mM glucose, 24 mM NaHCO₃ at pH 7.4) (Hakim C H, et al. Methods Mol Biol. 2011; 709:75-89). EDL and diaphragm muscles were equilibrated for 10 min before determining optimal length (Lo) and supramaximal output (120% stimulating voltage) as in Wei-LaPierre, L. et al. (2013).

Muscles were then subjected to a twitch warm-up protocol (three 200 ms 1 Hz stimuli separated by 20 sec), and tetanus warm up protocol (three 500 ms, 150 Hz stimuli separated by 1 min) before frequency dependence was determined. Frequency dependence was determined with increasing 500 ms stimulations separated by one minute unless noted otherwise. For EDL, the stimulations were as follows, 1 Hz (200 ms), 25 Hz, 50 Hz, 75 Hz, 100 Hz, 125 Hz, 150 Hz, 175 Hz, 200 Hz, and 250 Hz. For diaphragm, stimulations were 1 Hz (200 ms), 10 Hz, 20 Hz, 40 Hz, 60 Hz, 80 Hz, 100 Hz, 125 Hz, 150 Hz, and 200 Hz. Muscle force was recorded using 610A Dynamic Muscle Control LabBook software (Aurora Scientific) and analyzed using Clampfit 10.0 software. Specific force was calculated using a wet weight of the muscle and optimized length between the proximal and distal myotendinous junctions (Hakim C H, et al. Methods Mol Biol. 2011; 709:75-89, and Hakim C H, et al. J Vis Exp. 2013(72)). Two-way ANOVA analysis was used to determine statistical differences between groups for frequency dependence.

### Myotonia

Myotonia was measured using in vitro muscle contraction. EDLs were isolated from 20-week WT and Caᵥ1.1^{Δe29} mice and optimized as in the previous section. To obtain a baseline for the muscle without the presence on myotonia, muscles were equilibrated for 25 minutes in Ringer's media. Muscles were then subject to a protocol to determine myotonia with 3 successive 200 ms 1 Hz twitches separated by 20 seconds, a 3-minute rest period, then 3 successive 500 ms 150 Hz tetani separated by 3 minutes. Ringer's media with 100 uM 9-Acridinecarboxylic acid (9-AC) flowed into the bath for 25 minutes, and the myotonia protocol was repeated. This timing was to ensure enough time for buffer exchange and for the muscle to equilibrate in the new bath. To determine the impact of verapamil on myotonia in WT and Caᵥ1.1^{Δe29} muscle, the myotonia protocol (noted earlier in this section) was performed in the presence of Ringer's media containing either 5 μM or 20 μM verapamil then followed by Ringer's media containing 100 μM 9-AC and either 5 μM or 20 μM. Muscle force was recorded using 610A Dynamic Muscle Control LabBook software (Aurora Scientific), and traces were analyzed using Clampfit 10.0 software to calculate the area under the curve. The area under the curve was then normalized to the specific force (calculated as in the previous section) and used for statistical analysis.

### Transient Weakness

Transient weakness was measured in EDL muscle with in vitro muscle contraction. EDLs were isolated and optimized from 20-week-old WT or Caᵥ1.1^{Δe29} mice, as mentioned in previous sections. The experiments were contralaterally controlled where one side was equilibrated in normal Ringer's media, while the other side was equilibrated in Ringer's media with 100 μM 9-AC for at least 25 minutes. Muscles were then subjected to a transient weakness protocol of 45 successive 500 ms 100 Hz tetani separated by 4 seconds. To determine the impact of different calcium channel blockers on transient weakness, the drug was delivered via the Ringer's media in a contralateral fashion where one side received just the drug, and the other was the drug plus 9-AC. The calcium channel blockers tested were verapamil at a concentration of both 5 μM and 20 μM, Nifedipine at 75 nM, Amlodipine at 10 μM, and Diltiazem at 10 μM. For all drugs tested, the muscles were equilibrated in the drugged media for 25 minutes before performing the transient weakness protocol. Muscle force was recorded and analyzed as in previous section. Specific force was calculated, and for statistical analysis, we also looked as the percent of the initial force generated.

### Statistical Analysis

All data average data represent shows the means S.E.M. represented either as a bar with error bars or as a closed circle with error bands. Data points collected for a single mouse were represented as open circles (one mouse is n=1); however, for in vitro muscle contraction techniques, open circles represent one muscle (one muscle is n=1). The number of sampled units, n, upon which we reported statistics, is the single mouse for the in vivo experiments (one mouse is n=1). GraphPad Prism 9 software was used for statistical analyses. One-way or Two-way ANOVA with multiple comparisons was used to test for significant differences between each of the groups tested. For survival, each group was compared to each other group Kaplan-Meier log-rank test. For all figures, *P<0.05, **P<0.01, ***P<0.001, and ****P<0.0001 were used.

### Example 2

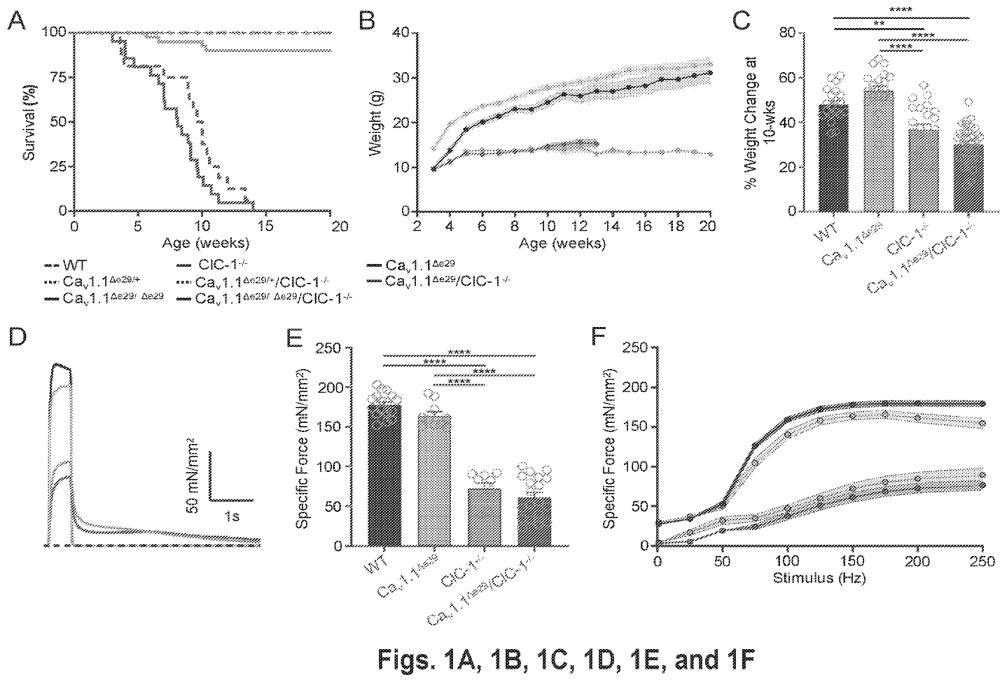

As shown in FIG. 1, Caᵥ1.1^{Δe29} and ClC-1^{-/-} alleles exhibit synthetic lethality, and result in significantly reduced body weight and severe muscle weakness in mice. FIG. 1A shows the results Kaplan-Meier survival analysis of WT (n=10), Caᵥ1.1^{Δe29/+} (n=13), Caᵥ1.1^{Δe29} (n=15), ClC-1^{-/-} (n=40), Caᵥ1.1^{Δe29/+}/ClC-1^{-/-} (n=16), and Caᵥ1.1^{Δe29/Δe29}/ClC-1^{-/-} (n=21). Survival in both Caᵥ1.1^{Δe29/+}/ClC-1^{-/-} and Caᵥ1.1^{Δe29/Δe29}/ClC-1^{-/-} mice is significantly reduced compared to WT mice. This reduction in survival is significantly reduced compared to ClC-1^{-/-} alone mice as well. Caᵥ1.1^{Δe29/+}/ClC-1^{-/-} and Caᵥ1.1^{Δe29/Δe29}/ClC-1^{-/-} mice do not survive beyond 14-weeks of age and both have median life expectancies of 9.8-weeks and 8.1-weeks respectively. FIG. 1B shows the results of weekly body weight analysis. FIG. 1C shows percent body weight change from weaning at 10 weeks. Together, FIGS. 1B-C show that Caᵥ1.1^{Δe29}/ClC-1^{-/-} mice have a failure to thrive compared to WT mice. However, week-to-week body weight and percent body weight change of Caᵥ1.1^{Δe29}/ClC-1^{-/-} mice are comparable to that of ClC-1^{-/-} alone mice. FIG. 1D shows representative specific force traces, and FIG. 1E shows average peak specific force elicited by 150 Hz (500 ms) tetanic stimulation of isolated EDL muscles from 10-wk mice. FIG. 1F shows average frequency dependence of specific force generation, elicited from isolated EDL muscle from 10-wk mice. The EDL muscle is Caᵥ1.1^{Δe29}/ClC-1^{-/-} is very weak. Symbols, open circles, individual mice; bars and closed circles, means±SEM. Altogether from FIGS. 1D, 1E, and 1F, the Caᵥ1.1^{Δe29}/ClC-1^{-/-} mice show a significant force generation deficit compared to that of WT muscle.

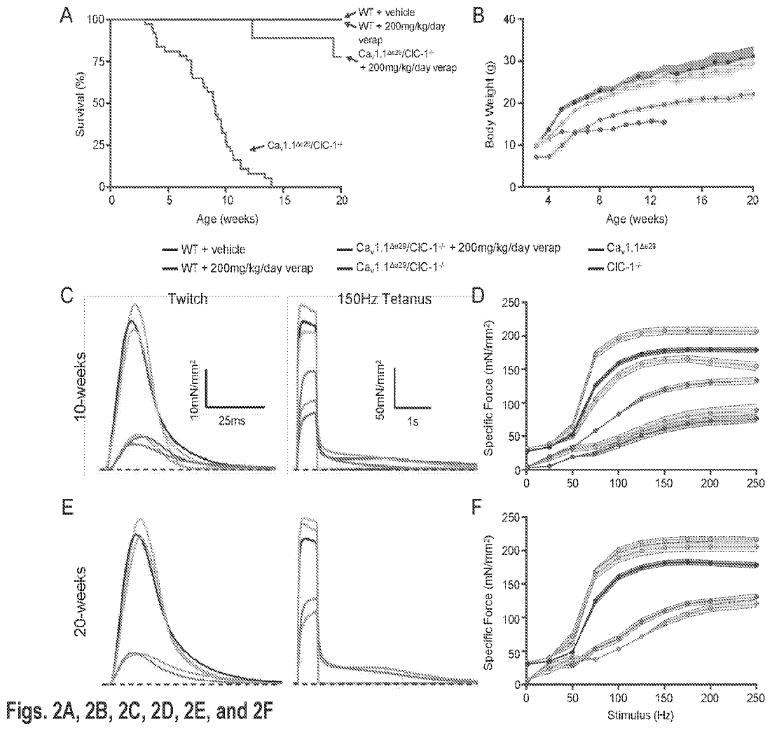

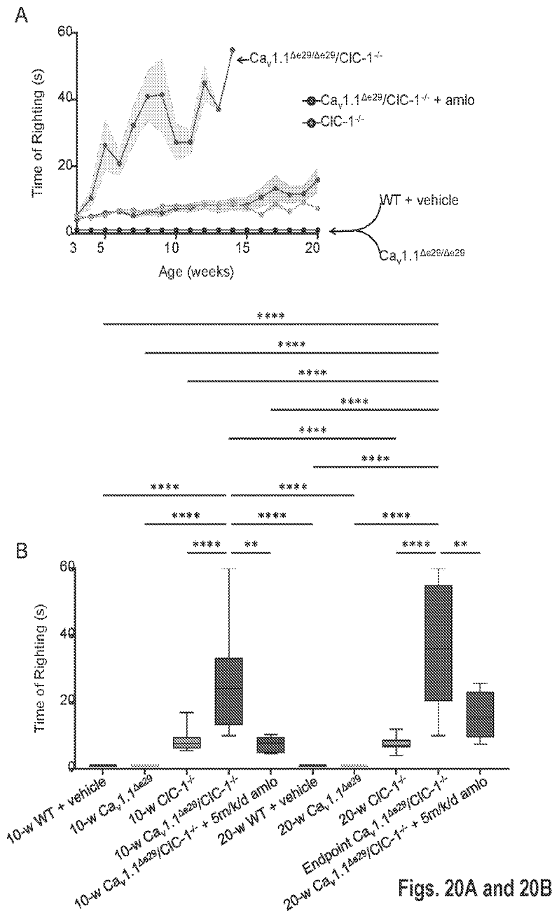

Next, the effects of verapamil in increasing survival, body weight, and muscle function in Caᵥ1.1^{Δe29}/ClC-1^{-/-} mice were investigated. As shown in FIG. 2, verapamil treatment improves survival, body weight, and muscle function in Caᵥ1.1^{Δe29}/ClC-1^{-/-} mice. FIG. 2A shows the results of the Kaplan-Meier survival analysis of WT+vehicle (n=10), WT+200 mg/kg/day verapamil (n=10), Caᵥ1.1^{Δe29}/ClC-1^{-/-} (n=37), and Caᵥ1.1^{Δe29/Δe29}/ClC-1^{-/-}+verapamil (n=9). Verapamil is dosed in mouse nutrition/hydration food cups. From this panel, treatment with 200 mg/kg/day of verapamil significantly improves lifespan in the Caᵥ1.1^{Δe29}/ClC-1^{-/-} mice. Terminal experimentation was complete after 20-weeks of age for the mice that survived though the time course of the survival study. The lifespan of verapamil treated Caᵥ1.1^{Δe29}/ClC-1^{-/-} mice was significantly extended compared to untreated Caᵥ1.1^{Δe29}/ClC-1^{-/-} remained robust. FIG. 2B shows the results of body weight analysis. Caᵥ1.1^{Δe29}/ClC-1^{-/-} mice treated with 200 mg/kg/day verapamil showed a significant improvement to weight gain compared to untreated Caᵥ1.1^{Δe29}/ClC-1^{-/-} mice. Treatment with verapamil in WT mice had no significant impact on body weight pointing towards verapamil treatment being safe for use in WT mice. FIGS. 2C and 2E show representative specific force traces elicited by twitch (left) and 150 Hz (500 ms) tetanic (right) stimulation of EDL muscle isolated from 10-wk (FIG. 2C) and 20-wk (FIG. 2E) mice. FIGS. 2D and 2F show a plot of average stimulation frequency dependence of specific force generation from isolated EDL muscles at 10 weeks (FIG. 2D) and 20 weeks (FIG. 2F) of age in the indicated genotype and treatment groups. From the in vitro muscle contraction data presented in FIG. 2C-D, there is significant improvement in force generation at 10-weeks in verapamil treated Caᵥ1.1^{Δe29}/ClC-1^{-/-} EDL muscle compared to untreated. This is due to a myo-protective impact of verapamil treatment as these experiments are done with no verapamil in the bath. The improvement seen at this age is thus due to the long-term verapamil treatment. Due to the premature death of the untreated Caᵥ1.1^{Δe29}/ClC-1^{-/-} mice, there is not a direct comparison that can be made at 20-weeks for the verapamil treated Caᵥ1.1^{Δe29}/ClC-1^{-/-} mice. Symbols, closed circles, means±SEM.

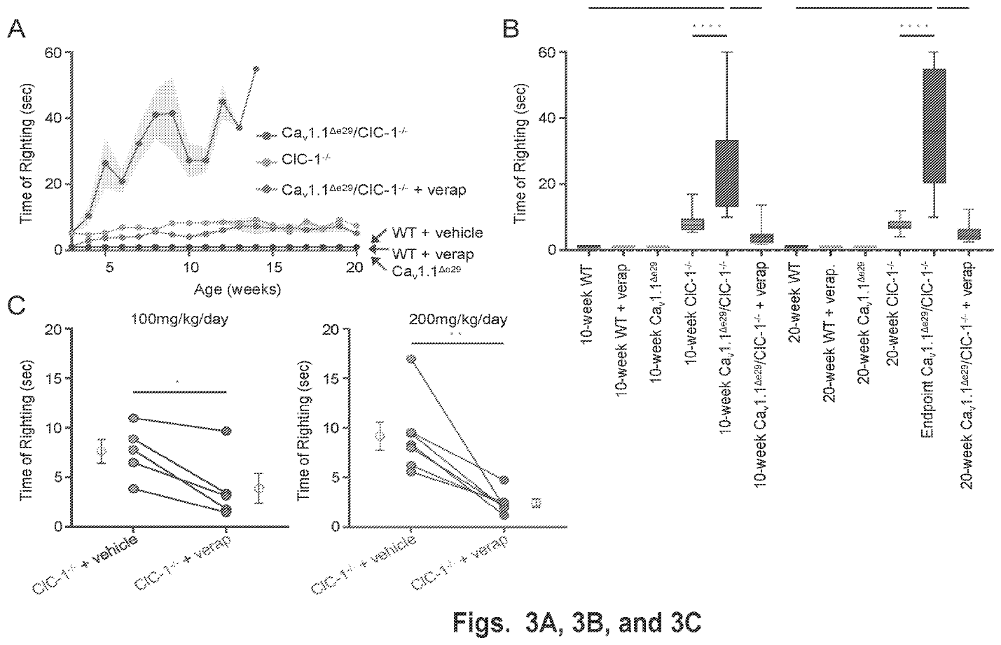

The effects of verapamil in improving the time of righting reflex in ClC-1^{-/-} and Caᵥ1.1^{Δe29}/ClC-1^{-/-} mice were also investigated. As shown in FIG. 3, verapamil treatment significantly improves the time of righting reflex in ClC-1^{-/-} and Caᵥ1.1^{Δe29}/ClC-1^{-/-} mice. FIG. 3A shows the results of weekly time of righting reflex analysis of Caᵥ1.1^{Δe29}/ClC-1^{-/-}, ClC-1-Caᵥ1.1^{Δe29}, WT, Caᵥ1.1^{Δe29}/ClC-1^{-/-}+verapamil and WT+verapamil mice, FIG. 3B shows average time of righting reflex in vehicle and verapamil treated mice at 10- and 20-week of age. Untreated Caᵥ1.1^{Δe29}/ClC-1^{-/-} mice do not survive to 20-week of age; therefore, the last recording before death is documented. Box notes Q1, median, and Q3; whiskers show minimum and maximum. From FIGS. 3A and 3B, there is a significant reduction in the time of righting relax when comparing untreated versus verapamil treated Caᵥ1.1^{Δe29}/ClC-1^{-/-} mice. Untreated Caᵥ1.1^{Δe29}/ClC-1^{-/-} mice will fail (take longer than 60 seconds to right) as they age. Additionally, their time to righting is significantly longer than that of CLC-1^{-/-} alone mice. Not only is the time of righting significantly reduced in the verapamil treated Caᵥ1.1^{Δe29}/ClC-1^{-/-} mice, but they also maintain a consistent time of righting even as they age. The improvement to time of righting seen with the Caᵥ1.1^{Δe29}/ClC-1^{-/-} mice is so much so that there is not a significant difference in the time of righting compared to that of WT mice which right in less than 1 second (recorded as “1”). FIG. 3C shows paired before (light circles) an after (dark circles) 2-wk verapamil treatment of ClC-1^{-/-} mice at 100 mg/kg/day (left) and 200 mg/kg/day (right) dosing in nutrition/hydration food cups. Though ClC-1−/− alone mice have WT Caᵥ1.1, verapamil was able to significantly reduce time of righting for both dosages tested in these paired experiments. This points towards verapamil being a potential therapeutic for myotonia congenita. Symbols, closed circles, individual mice; open circles, means±SEM.

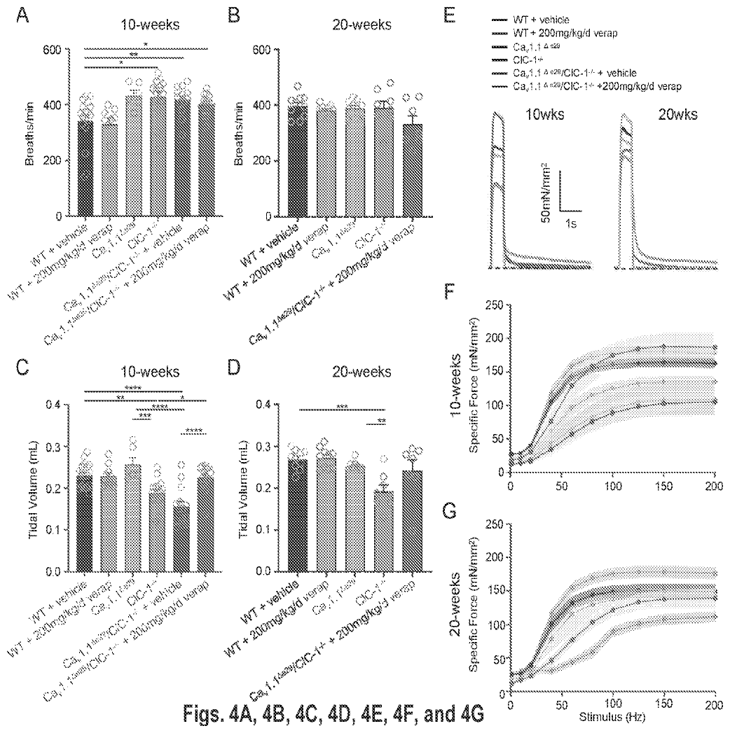

The effects of verapamil in improving respiratory function and diaphragm strength in ClC-1^{-/-} and Caᵥ1.1^{Δe29}/ClC-1^{-/-} mice were investigated. As shown in FIG. 4, verapamil treatment significantly improves respiratory function and diaphragm strength in Caᵥ1.1^{Δe29}/ClC-1^{-/-} mice. Whole-body plethysmography for 10-wk (FIGS. 4A and 4C) and 20-wk (FIGS. 4B and 4D) old mice in the indicated genotype and treatment groups. FIGS. 4A and 4B show the frequency of respiration (breaths/min), and FIGS. 4C and 4D show tidal volume of respiration (mL). Overall, Caᵥ1.1^{Δe29}/ClC-1^{-/-} mice treated with verapamil show no significant difference in frequency or tidal volume at 20-weeks. At 10-weeks, tidal volume is significantly improved with verapamil treatment in Caᵥ1.1^{Δe29}/ClC-1^{-/-} mice compared to untreated. The tidal volume in treated Caᵥ1.1^{Δe29}/ClC-1^{-/-} mice returns to that of WT mice at 10-weeks. FIG. 4E shows representative tetanic (150 Hz, 500 ms) specific force traces from diaphragm strips isolated from 10-wk (left) and 20-wk (right) old mice in the indicated genotypes and treatment groups. FIGS. 4F and 4G show plots of average stimulation frequency dependence of specific force generated from diaphragm strips isolated from 10-wk (FIG. 4F) and 20-wk (FIG. 4G) old mice in the indicated genotypes and treatment groups. From FIGS. 4E, 4F, and 4G, there is a significant improvement in diaphragm strip muscle force and frequency dependence in the verapamil treated Caᵥ1.1^{Δe29}/ClC-1^{-/-} mice. With the verapamil treatment, the Caᵥ1.1^{Δe29}/ClC-1^{-/-} mice show WT strength in their diaphragm strips for both 10- and 20-weeks. Further, the improvement seen in frequency depended at 10-weeks is robust when comparing the untreated to the treated Caᵥ1.1^{Δe29}/ClC-1^{-/-} muscle. When relating the construction data to the plethysmography data presented in this figure, there is an overall deficit in respiratory function in the Caᵥ1.1^{Δe29}/ClC-1^{-/-} mice that is significantly improved with verapamil treatment. Symbols, open circles, individual mice; bars and closed circles, means±SEM. Note: Untreated Caᵥ1.1^{Δe29}/ClC-1^{-/-} mice do not survive to 20-week of age.

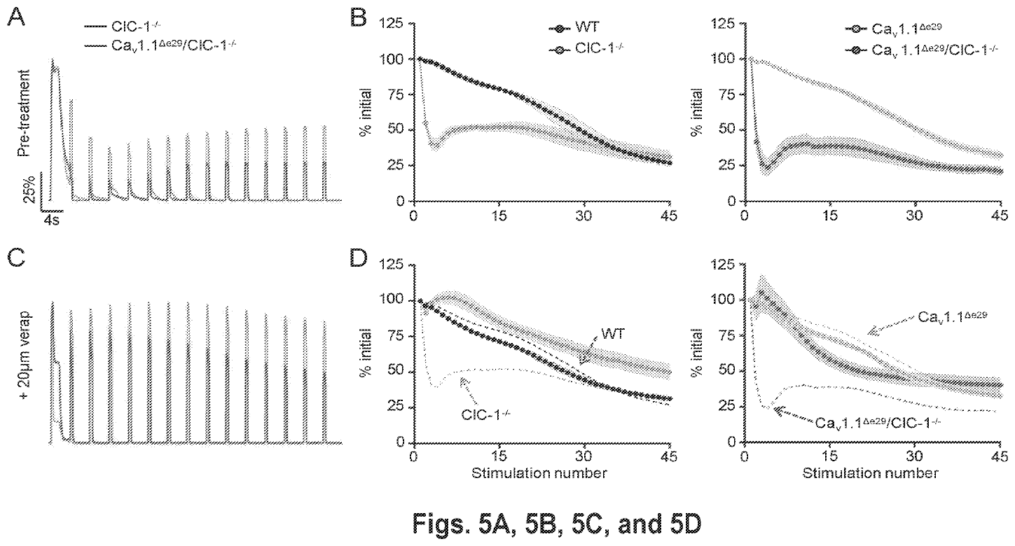

The effects of verapamil in improving severe transient muscle weakness in ClC-1^{-/-} and Caᵥ1.1^{Δe29}/ClC-1^{-/-} mice were investigated. As shown in FIG. 5, Caᵥ1.1^{Δe29}/ClC-1^{-/-} muscle exhibits severe transient weakness that is significantly improved by the addition of verapamil. FIGS. 5A and 5C show normalized representative force traces of the first 15 tetani (100 Hz, 500 ms) separated by four seconds, recorded ex vivo from EDLs isolated from 6-wk ClC-1^{-/-} and Caᵥ1.1^{Δe29}/ClC-1^{-/-} mice in the absence (FIG. 5A) and presence (FIG. 5C) of 20 μM verapamil added to the bath. FIGS. 5B and 5D show plots of the average peak tetanic EDL forces normalized to the initial stimulus, elicited by 44 subsequent 100 Hz, 500 ms tetanic stimulations separated by four seconds from 6-wk WT (n=4), ClC-1^{-/-} (n=4), Caᵥ1.1^{Δe29} (n=4) and Caᵥ1.1^{Δe29}/ClC-1^{-/-}, n=4) mice in the absence (FIG. 5B) and presence (FIG. 5D) of 20 μM verapamil added to the bath for ClC-1^{-/-} (n=4) and Caᵥ1.1^{Δe29}/ClC-1^{-/-}, n=4) EDLs. Dashed lines in FIG. 5D represent average data presented in FIG. 5B as a reference for pre-treatment. Overall, this figure show that there is a severed transient weakness in Caᵥ1.1^{Δe29}/ClC-1^{-/-} EDL muscle. The transient weakness seen is more severe than that in ClC-1^{-/-} alone EDL muscle. With verapamil treatment, we see a significant improvement in the transient weakness in both the ClC-1^{-/-} and Caᵥ1.1^{Δe29}/ClC-1^{-/-} EDL muscle. Verapamil's presence in the bath, decreased the force drop in both ClC-1^{-/-} and Caᵥ1.1^{Δe29}/ClC-1^{-/-} EDL muscle and there is a significant improvement in the recovery from transient weakness, Symbols, closed circles, mean±SEM.

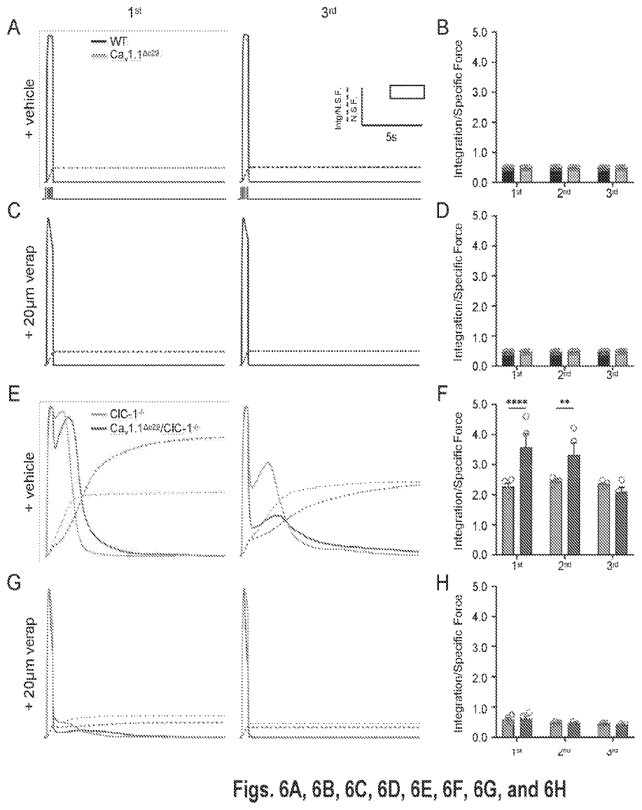

The effects of verapamil in reducing myotonia in ClC-1^{-/-} and Caᵥ1.1^{Δe29}/ClC-1^{-/-} mice were investigated. As shown in FIG. 6, verapamil significantly reduces myotonia in both Caᵥ1.1^{Δe29}/ClC-1^{-/-} and ClC-1^{-/-} mouse muscle. FIGS. 6A and 6C show normalized representative specific force traces of the first (left) and third (right) tetani (150 Hz, 500 ms) from EDLs isolated from 6-wk WT and Caᵥ1.1^{Δe29} mice in the absence (FIG. 6A) and presence (FIG. 6C) of 20 mM verapamil added to the bath. Dashed lines represent accumulated force. FIGS. 6B and 6D show average normalized integration of force for WT and Caᵥ1.1^{Δe29} EDLs across 3 tetanic stimulations (150 Hz, 500 ms) in the absence (FIG. 6B) and presence (FIG. 6D) of 20 mM verapamil added to the bath. FIGS. 6E and 6G show normalized representative specific force traces of the first (left) and third (right) tetani (150 Hz, 500 ms) from EDLs isolated from 6-wk ClC-1^{-/-} and Caᵥ1.1^{Δe29}/ClC-1^{-/-} mice in the absence (FIG. 6E) and presence (FIG. 6G) of 20 mM verapamil added to the bath. Dashed lines represent accumulated force. FIGS. 6F and 6H show average integration of force for ClC-1^{-/-} and Caᵥ1.1^{Δe29}/ClC-1^{-/-} EDLs across 3 tetanic stimulations (150 Hz, 500 ms) in the absence (FIG. 6B) and presence (FIG. 6D) of 20 mM verapamil added to the bath. Overall, this figure shows that the presence of Caᵥ1.1^{Δe29} significantly prolongs myotonia in vitro compared to the myotonia seen in ClC-1^{-/-} alone muscle. Verapamil's presence in the bath significantly reduces the myotonia seen in both ClC-1^{-/-} and Caᵥ1.1^{Δe29}/ClC-1^{-/-} EDL muscle. Symbols, open circles, individual mice; bars, mean±SEM. Contralateral EDLs were used for each untreated and treated experiment. All traces plotted with the same scale of time and normalized force for comparison.

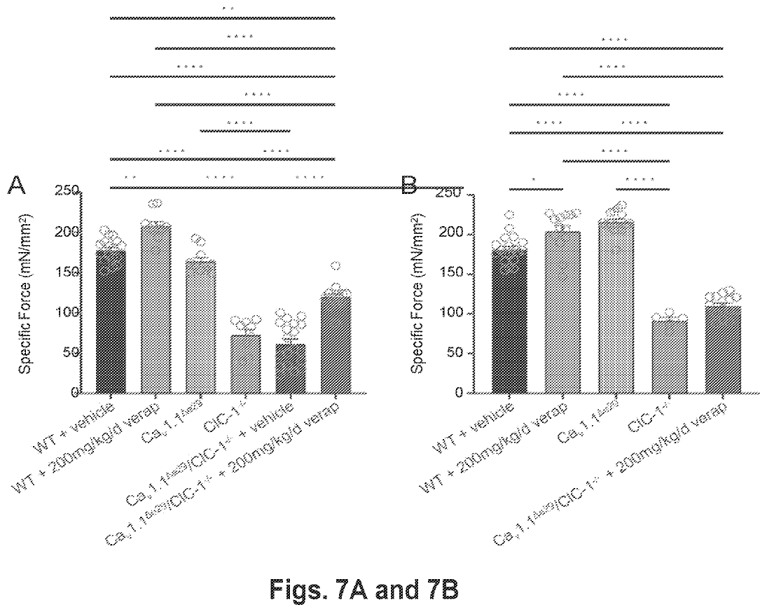

The effects of verapamil in rescuing EDL muscle force generation in ClC-1^{-/-} and Caᵥ1.1^{Δe29}/ClC-1^{-/-} mice were investigated. As shown in FIG. 7, long term verapamil oral feeding of Caᵥ1.1^{Δe29}/ClC-1^{-/-} mice results in significant rescue of EDL muscle force generation. Average peak specific force elicited using a 150 Hz (500 ms) tetanus stimulus of EDLs isolated from 10-wk (FIG. 7A) and 20-wk (FIG. 7B) mice of indicated genotype and treatment. At 10-weeks, the untreated Caᵥ1.1^{Δe29}/ClC-1^{-/-} muscle shows a significant force generation deficit at 10-week. There is a significant improvement in force generation in the treated Caᵥ1.1^{Δe29}/ClC-1^{-/-} EDL muscle compared to that on untreated. The improvement seen is hypothesized to be a myo-protective impact of verapamil treatment since no verapamil is present in the bath at the time of the experimentation. Symbol, open circles, individual mice; bars, mean±SEM.

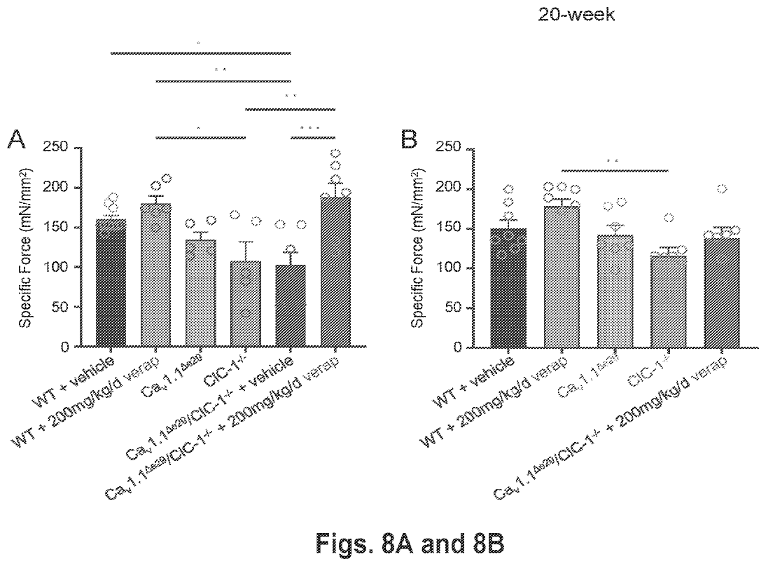

The effects of verapamil in rescuing iaphragm muscle force generation in ClC-1^{-/-} and Caᵥ1.1^{Δe29}/ClC-1^{-/-} mice were investigated. As shown in FIG. 8, long term verapamil oral feeding of Caᵥ1.1^{Δe29}/ClC-1^{-/-} mice results in significant rescue of diaphragm muscle force generation. Average peak specific force elicited using a 150 Hz (500 ms) tetanus stimulus of diaphragm strips isolated from 10-wk (FIG. 8A) and 20-wk (FIG. 8B) mice of indicated genotype and treatment. At 10-weeks, the untreated Caᵥ1.1^{Δe29}/ClC-1^{-/-} diaphragm muscle showed a significant force generation deficit at 10-week. There is a significant improvement in force generation in the treated Caᵥ1.1^{Δe29}/ClC-1^{-/-} diaphragm strip compared to that of untreated. The improvement seen is due to a myo-protective impact of verapamil treatment since no verapamil is present in the bath at the time of the experimentation. This rescue of force production by verapamil is not a significantly difference between treated Caᵥ1.1^{Δe29}/ClC-1^{-/-} diaphragm strip and WT. This improvement is maintained at 20-weeks of age as well. Symbol, open circles, individual mice; bars, mean±SEM.

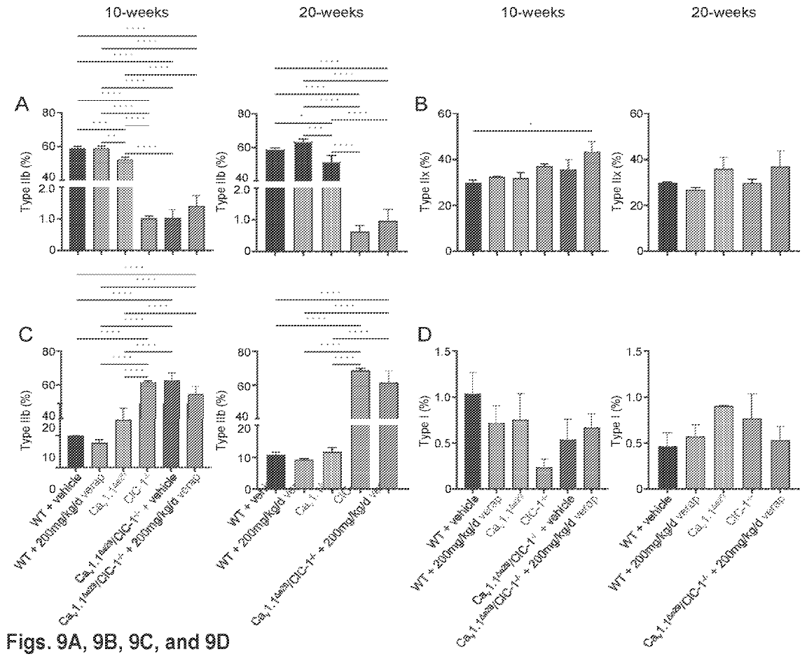

FIGS. 9A, 9B, 9C, and 9D are a set of graphs showing the results of quantification and statistical analysis of fiber type distribution of tibialis anterior muscle. Quantification of type IIb (FIG. 9A), type Ix (FIG. 9B), type IIa (FIG. 9C), and type I (FIG. 9D) fibers for 10-wk (left) and 20-wk (right) tibialis anterior (n=5/group) are shown. The fiber type distributions of the untreated and treated Caᵥ1.1^{Δe29}/ClC-1^{-/-} tibialis anterior is dominated by the phenotype shift seen with the presence of ClC-1^{-/-} at both 10- and 20-weeks-of-age.

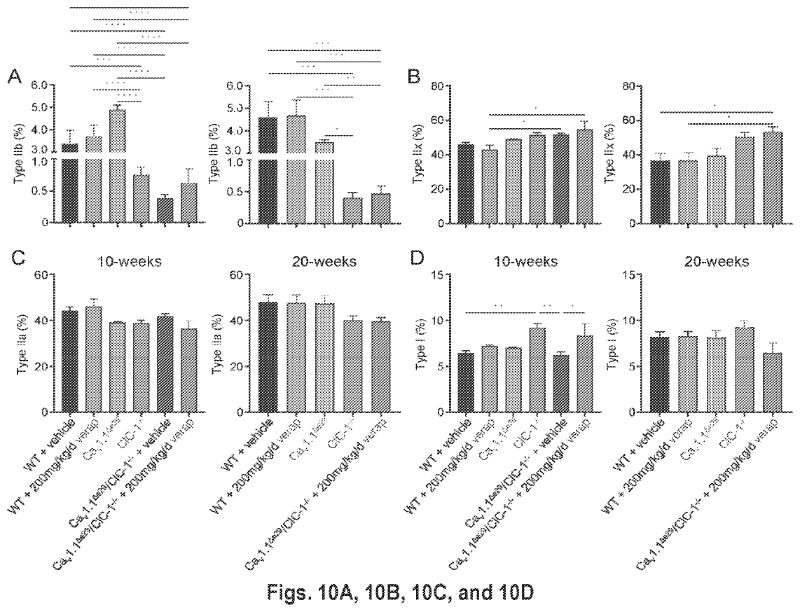

FIGS. 10A, 10B, 10C, and 10D are a set of graphs showing the results of quantification and statistical analysis of fiber type distribution of diaphragm muscle. Quantification of type IIb (FIG. 10A), type IIx (FIG. 10B), type IIa (FIG. 10C), and type I (FIG. 10D) fibers for 10-wk (left) and 20-wk (right) diaphragm (n=5/group). The fiber type distributions of the untreated and treated Caᵥ1.1^{Δe29}/ClC-1^{-/-} diaphragm is dominated by the phenotype shift seen with the presence of ClC-1^{-/-} at both 10- and 20-weeks-of-age.

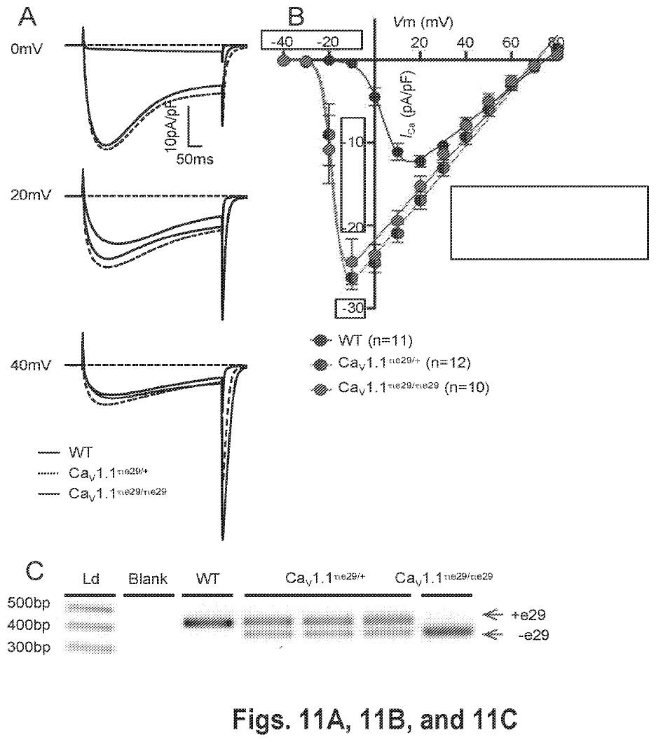

FIGS. 11A, 11B, and 11C are a set of diagrams showing that heterozygous and homozygous Caᵥ1.1^{Δe29} mice exhibit similar Caᵥ1.1 voltage-dependence and peak current densities in flexor digitorum brevis muscle. FIG. 11A shows representative current density traces from whole cell patch clamp of flexor digitorum brevis fibers isolated from 4-week WT, Caᵥ1.1^{Δe29/+} (dashed), and Caᵥ1.1^{Δe29/Δe29} (solid) mice at 0 mV (top), +20 mV (middle) and +40 mV (bottom). FIG. 11B shows a plot of average current-voltage relationship of Caᵥ1.1 activity measured in WT, Caᵥ1.1^{Δe29/+} (circles, dashed), and Caᵥ1.1^{Δe29/Δe29} (circle, solid line) flexor digitorum brevis fibers isolated from 4-wk mice. Together, FIGS. 11A-B show that heterozygous loss of exon 29 in Caᵥ1.1 has a near identical current density and current-voltage relationship to that of the homozygous loss of exon 29. FIG. 11C shows RT-PCR products of Ca 1.1 RNA isolated from tibialis anterior from 10-wk mice. PCR amplifications are from exons 27 to 31 of Caca1ns cDNA.

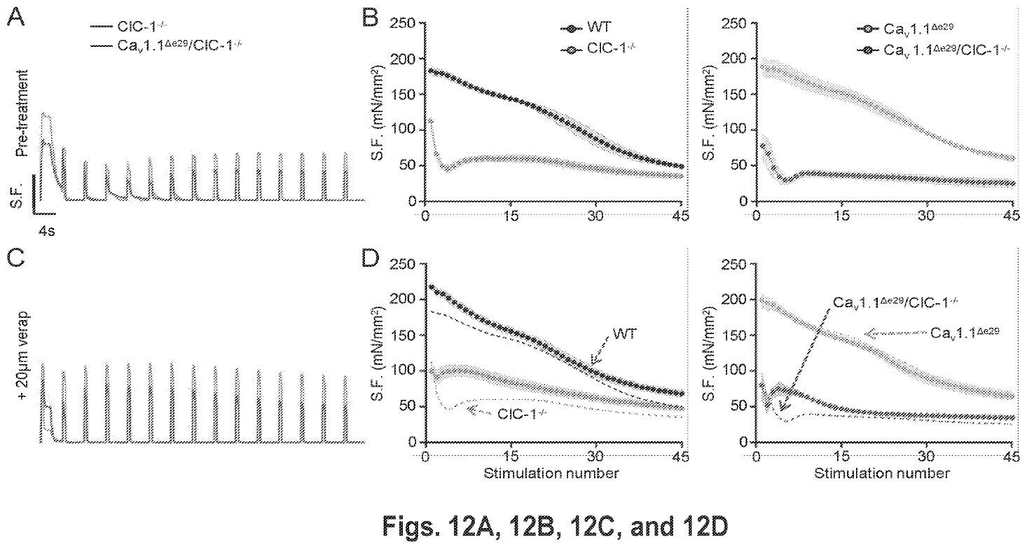

FIGS. 12A, 12B, 12C, and 12D are a set of graphs showing that Caᵥ1.1^{Δe29}/ClC-1^{-/-} muscle exhibits severe transient weakness that is significantly improved by the addition of verapamil (not normalized to the first peak force). FIGS. 12A and 12C show representative specific force traces of the first 15 tetani (100 Hz, 500 ms) separated by four seconds, recorded ex vivo from EDLs isolated from 6-wk ClC-1^{-/-} and Caᵥ1.1^{Δe29}/ClC-1^{-/-} mice in the absence (FIG. 12A) and presence (FIG. 12C) of 20 μM verapamil added to the bath. FIGS. 12B and 12D show a plot of the average peak tetanic EDL, elicited by 44 subsequent 100 Hz, 500 ms tetanic stimulations separated by four seconds from 6-wk WT (n=4), ClC-1^{-/-} (n=4), Caᵥ1.1^{Δe29} (n=4) and Caᵥ1.1^{Δe29}/ClC-1^{-/-} n=4) mice in the absence (FIG. 12B) and (FIG. 12D) presence of 20 μM verapamil added to the bath for ClC-1^{-/-} (n=4) and Caᵥ1.1^{Δe29}/ClC-1^{-/-} n=4) EDLs. Dashed lines in FIG. 12D represent average data presented in FIG. 12B as a reference for pre-treatment. Findings in this figure are similar to that of FIG. 5. This figure furthers the severe transient weakness that is seen in the Caᵥ1.1^{Δe29}/ClC-1^{-/-} EDL muscle that is significantly improved with verapamil treatment. Symbols, closed circles, mean±SEM.

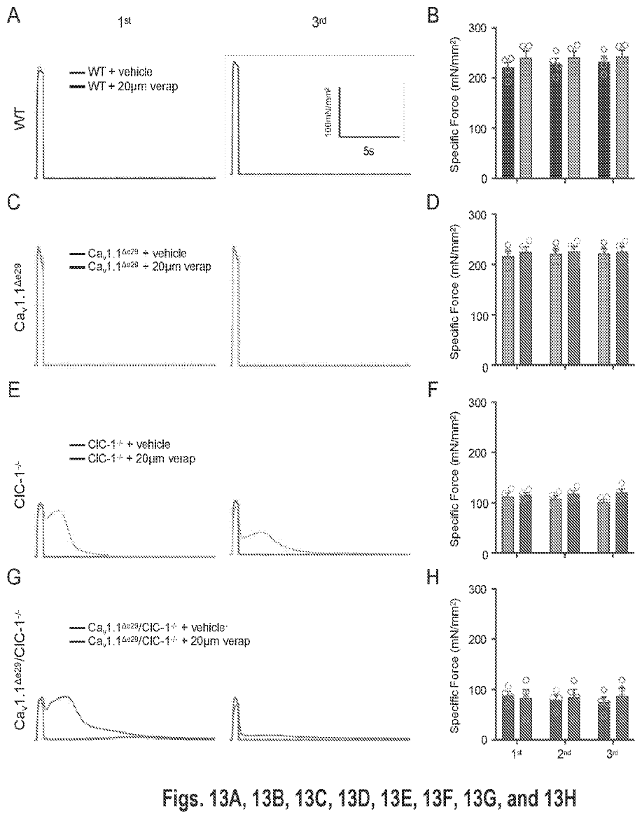

FIGS. 13A, 13B, 13C, 13D, 13E, 13F, 13G, and 13H are a set of graphs showing that verapamil treatment does not reduce peak contraction force of WT, Caᵥ1.1^{Δe29}, ClC-1^{-/-} and Caᵥ1.1^{Δe29}/ClC-1^{-/-} mouse muscle. FIGS. 13A, 13C, 13E, and 13G show representative traces of the first (left) and third (right) tetani (150 Hz, 500 ms) in WT (FIG. 13A), Caᵥ1.1^{Δe29} (FIG. 13C), ClC-1^{-/-} (FIG. 13E), and Caᵥ1.1^{Δe29}/ClC-1^{-/-} (FIG. 13G) EDLs in the absence and presence of 20 mM verapamil. Treatment is depicted in legends. FIGS. 13B, 13D, 13F, and 13H show average specific force for WT (FIG. 13B), Caᵥ1.1^{Δe29} (FIG. 13D), ClC-1^{-/-} (FIG. 13F), and Caᵥ1.1^{Δe29}/ClC-1^{-/-} (FIG. 13H) EDLs across 3 tetanic stimulations in the absence and presence of 20 mM verapamil. Treatment is depicted in legends. This experiment was performed to show that the presence of verapamil in the bath has no impact on the specific force generation of the EDL muscle for any of the groups tested. Symbols, open circles, individual mice; bars, mean, and SEM.

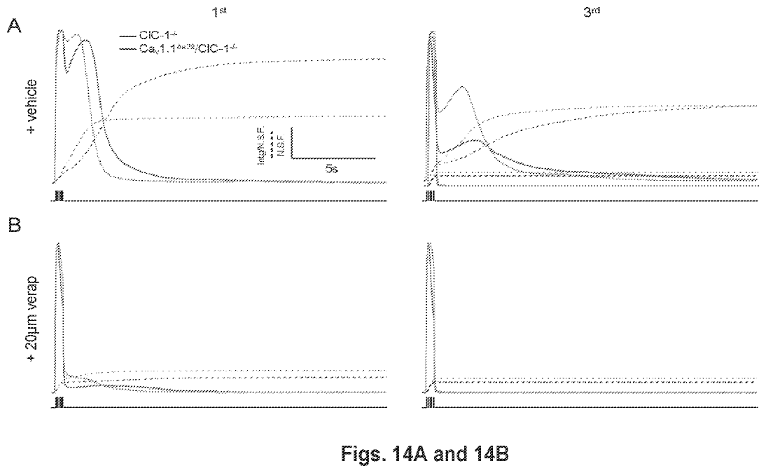

FIGS. 14A and 14B are a set of graphs showing that verapamil significantly reduces myotonia in both Caᵥ1.1^{Δe29}/ClC-1^{-/-} and ClC-1^{-/-} mouse muscle. FIG. 14A shows representative trace of normalized specific force generation of the first (left) and third (right) tetani (150 Hz, 500 ms) in ClC-1^{-/-} (solid) and Caᵥ1.1^{Δe29}/ClC-1^{-/-} (sold) EDL in the absence of 20 μM verapamil. Dashed lines represent accumulated force. FIG. 14B shows representative trace of the first (left) and third (right) tetani (150 Hz, 500 ms) in ClC-1^{-/-} (solid) and Caᵥ1.1^{Δe29}/ClC-1^{-/-} (solid) EDL in the presence of 20 μM verapamil. Dashed lines represent accumulated force. Note: Traces shown in FIG. 6 are replotted here with expanded timescales to better observe myotonia. Findings as noted in the section for FIG. 6 are further demonstrated with this alternate display.

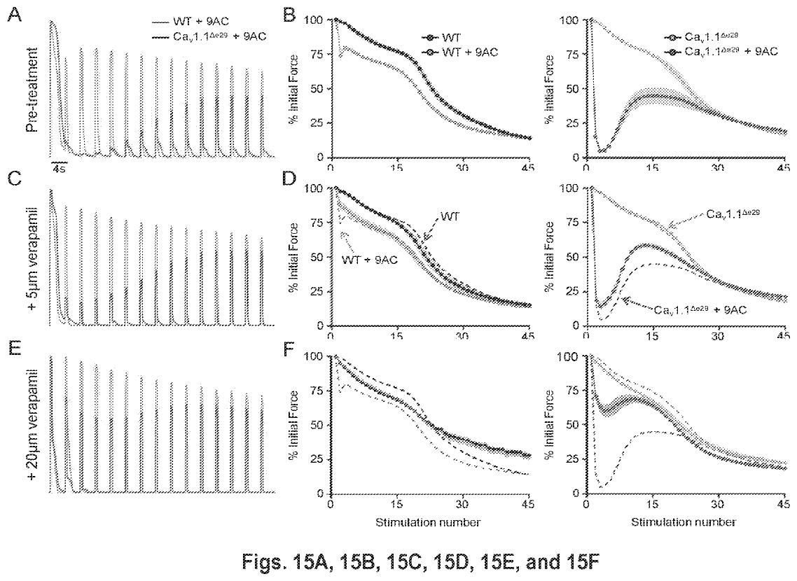

FIGS. 15A, 15B, 15C, 15D, 15E, and 15F are a set of graphs showing that Caᵥ1.1^{Δe29} exacerbates transient weakness in myotonic muscle and is alleviated by verapamil. FIG. 15A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). From this we can see that transient weakness in myotonic EDL is worsened with the absence of exon 29 in Ca 1.1. FIG. 15B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. To the right, the myotonic WT EDL muscle had a slight transient weakness that was quickly recovered. To the right, there is a significant initial force drop in the Caᵥ1.1^{Δe29}+9AC EDL and recovery from this transient weakness takes many stimulations. FIGS. 15C and 15E show normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 5 mM verapamil (FIG. 15C) or 20 mM verapamil (FIG. 15E) added to the bath. FIG. 15D shows a plot of the average peak tetanic EDL forces normalized to the initial stimulus, elicited by 44 subsequent 100 Hz, 500 ms tetanic stimulations separated by four seconds from WT+5 mM verapamil (n=5), WT+9-AC+5 mM verapamil (n=5), Caᵥ1.1^{Δe29}+5 mM verapamil (n=5) and Caᵥ1.1^{Δe29}+9-AC+5 mM verapamil (n=5) EDLs. FIG. 15F shows a plot of the average peak tetanic EDL forces normalized to the initial stimulus, elicited by 44 subsequent 100 Hz, 500 ms tetanic stimulations separated by four seconds from WT+20 mM verapamil (n=5), WT+9-AC+20 mM verapamil (n=5), Caᵥ1.1^{Δe29}+20 mM verapamil (n=5) and Caᵥ1.1^{Δe29}+9-AC+20 mM verapamil (n=5) EDLs. Dashed lines in FIGS. 15D and 15F represent average data presented in FIG. 15B as a reference for pre-treatment. Overall, verapamil at both Sum and 20 um improve transient weakness in WT+9-AC and Caᵥ1.1^{Δe29}+9-AC EDL muscle. The improvement is significant and robust for the Caᵥ1.1^{Δe29}+9-AC at the 20 um concentrations. Symbols, closed circles, mean±SEM. Note: Contralateral EDLs were used when possible.

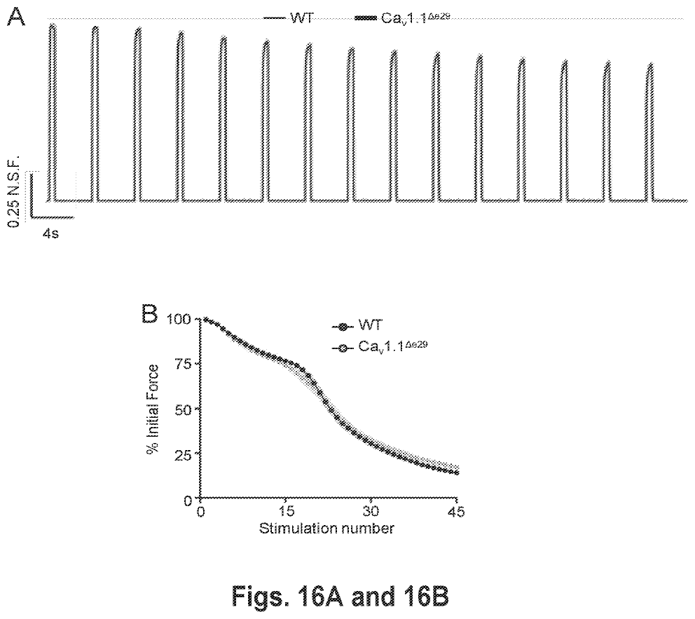

FIGS. 16A and 16B are a set of graphs showing that transient weakness is absent in WT and Caᵥ1.1^{Δe29} muscle. FIG. 16 shows representative traces of the first 15, tetani (150 Hz, 500 ms) separated by 4 seconds in 20-wk WT and Caᵥ1.1^{Δe29} EDL muscle. FIG. 16B shows a plot of the average peak tetanic EDL forces normalized to the initial stimulus, elicited by 44 subsequent 100 Hz, 500 ms tetanic stimulations separated by four seconds from EDLs isolated from 20-wk WT and Caᵥ1.1^{Δe29} mice. Symbols, closed circles, mean±SEM.

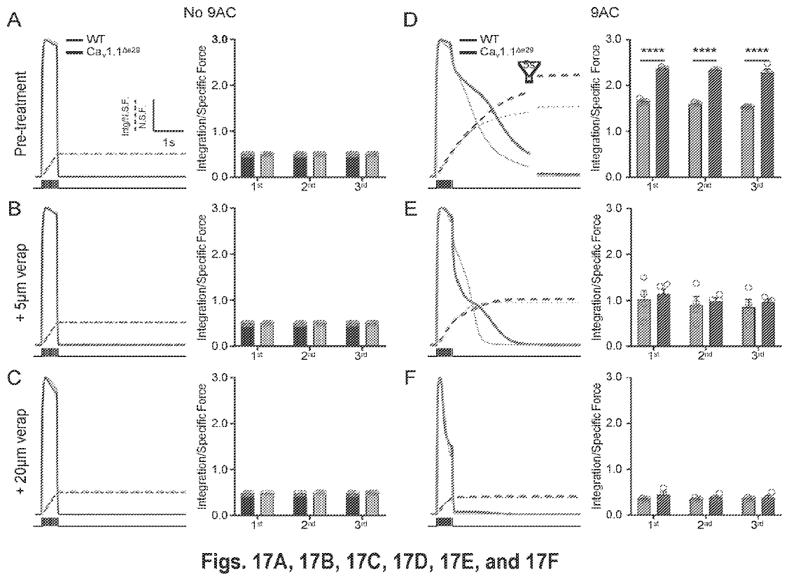

FIGS. 17A, 17B, 17C, 17D, 17E, and 17F are a set of graphs showing that Caᵥ1.1^{Δe29} significantly exacerbates myotonia. FIGS. 17A, 17B, and 17C (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 17A, left) and presence of 5 mM (FIG. 17B, left) and 20 mM (FIG. 17C, left) verapamil added to the bath (pre-treatment). Dashed lines represent accumulated force production. FIGS. 17A, 17B, and 17C (right) show a plot of average integration normalized to specific force depicted in respective left panels. FIGS. 17D, 17E, and 17F (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 17D, left) and presence of 5 mM (FIG. 17E, left) and 20 mM (FIG. 17F, left) verapamil added to the bath (pre-treatment). Dashed lines represent accumulated force production. FIGS. 17D, 17E, and 17F (right) show a plot of average integration normalized to specific force depicted in respective left panels. Symbols, open circles, individual mice; bars, mean and SEM. Note: Contralateral EDLs were used when possible.

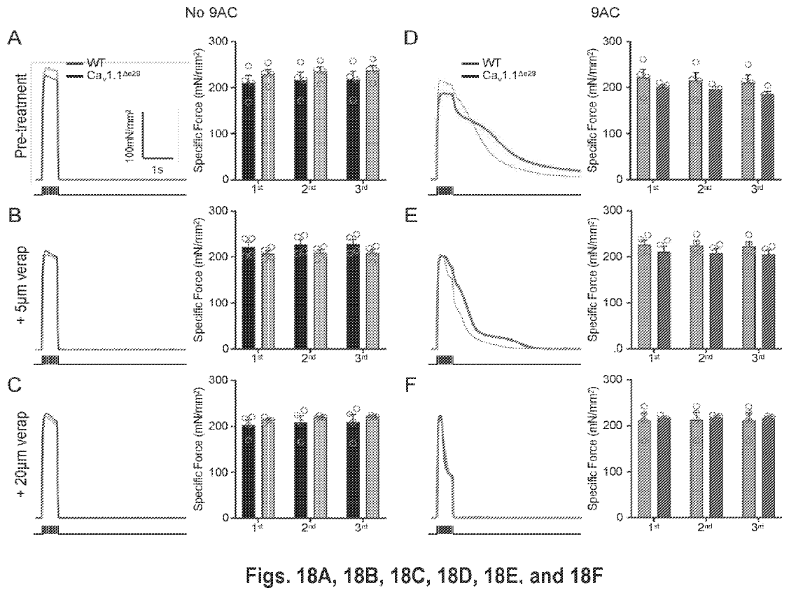

FIGS. 18A, 18B, 18C, 18D, 18E, and 18F are a set of graphs showing that verapamil treatment does not reduce peak contraction force of non-myotonic and myotonic WT and Caᵥ1.1^{Δe29} mouse muscle. Representative specific force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 18A, left) and presence of 5 mM (FIG. 18B, left) and 20 mM (FIG. 18C, left) verapamil added to the bath (pre-treatment). Dashed lines represent accumulated force production. FIGS. 18A, 18B, and 18C (right) show a plot of average integration of specific force depicted in respective left panels. FIGS. 18D, 18E, and 18F (left) show representative specific force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 18D, left) and presence of 5 mM (FIG. 18E, left) and 20 mM (FIG. 18F, left) verapamil added to the bath (pre-treatment). Dashed lines represent accumulated force production. FIGS. 18D, 18E, and 18F (right) show a plot of average integration of specific force depicted in respective left panels. Symbols, open circles, individual mice; bars, mean and SEM. Note: Contralateral EDLs were used when possible.

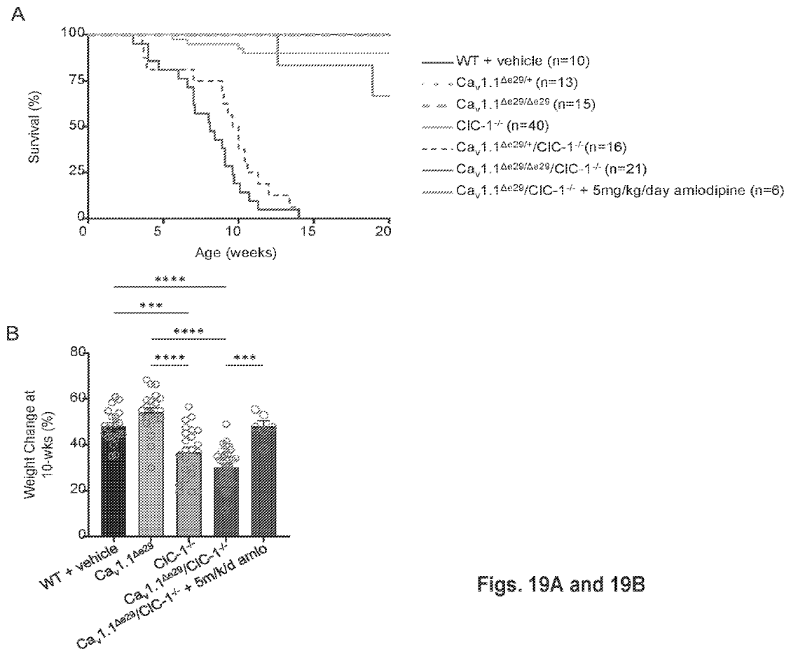

This example also provides the effects of amlodipine in the mouse model. As shown in FIG. 19, Caᵥ1.1^{De29} and CC-1^{-/-} alleles exhibit synthetic lethality, and result in significantly reduced body weight and severe muscle weakness in mice. Amlodipine treatment significantly improved survival and percent body weight change. FIG. 19A shows the results of Kaplan-Meier survival analysis of WT (n=10), Caᵥ1.1^{Δe29/+} (n=13), Caᵥ1.1^{Δe29} (n=15), ClC-1^{-/-} (n=40), Caᵥ1.1^{Δe29/+}/ClC-1^{-/-} (n=16), Caᵥ1.1^{Δe29/Δe29}/ClC-1^{-/-} (n=21), and Caᵥ1.1^{Δe29}/ClC-1^{-/-}+5 mg/kg/day amlodipine (n=6). Untreated Caᵥ1.1^{Δe29/+}/ClC-1^{-/-} and Caᵥ1.1^{Δe29/Δe29}/ClC-1^{-/-} mice have severely reduced lifespans; 14-weeks-of-age is the longest survival that the mice have reached, and they had a median life expectancy of only 8.9-weeks. The treatment of 5 mg/kg/day amlodipine, Caᵥ1.1^{Δe29}/ClC-1^{-/-} mice had a significantly improved survival. FIG. 19B show percent body weight change from weaning at 10 weeks. Caᵥ1.1^{Δe29}/ClC-1^{-/-} mice fail to thrive and show a significantly reduced body weight from that of WT mice, however this is not significantly different than the percent body weight change of ClC-1^{-/-} alone mice. Regardless, treatment with 5 mg/kg/day of amlodipine improve percent body weight change in Caᵥ1.1^{Δe29}/ClC-1^{-/-} mice that it was a significant improvement compared to untreated mice. The change in body weight gain with treatment was not significantly different than that of WT. Symbols, open circles, individual mice; bars and closed circles, means±SEM.

The effects of amlodipine in improving the time of righting reflex in Caᵥ1.1^{Δe29}/ClC-1^{-/-} mice was investigated. As shown in FIG. 20, amlodipine treatment significantly improves the time of righting reflex in Caᵥ1.1^{Δe29}/ClC-1^{-/-} mice compared to untreated. FIG. 20A shows weekly time of righting reflex analysis of Caᵥ1.1^{Δe29}/ClC-1^{-/-}, ClC-1^{-/-}, Caᵥ1.1^{Δe29}, WT, Caᵥ1.1^{Δe29}/ClC-1^{-/-}+amlodipine (purple) mice. Caᵥ1.1^{Δe29}/ClC-1^{-/-} mice have significantly longer time if righting reflex. As they age, the time to right increases until they reach failure to right. This is when the mouse takes longer than 60 seconds to right. Treatment with 5 mg/kg/day amlodipine in Caᵥ1.1^{Δe29}/ClC-1^{-/-} mice significantly improves time of righting. Week-to-week, there is no significant difference in righting in treated Caᵥ1.1^{Δe29}/ClC-1^{-/-} mice compared to ClC-1^{-/-} alone mice. FIG. 20B shows average time of righting reflex in vehicle and amlodipine treated mice at 10- and 20-week of age. This data further shows that Caᵥ1.1^{Δe29}/ClC-1^{-/-} mice have a significantly longer time of righting compared to WT mice as well as ClC-1^{-/-} alone mice. Treatment with 5 mg/kg/day of amlodipine significantly improve time of righting in Caᵥ1.1^{Δe29}/ClC-1^{-/-} mice compared to untreated Caᵥ1.1^{Δe29}/ClC-1^{-/-} mice. This improvement in the average time of righting is so much so that there is not a significant difference in time of righting between treated Caᵥ1.1^{Δe29}/ClC-1^{-/-} mice and WT mice. Note: Untreated Caᵥ1.1^{Δe29}/ClC-1^{-/-} mice do not survive to 20-week of age, therefore the last recording before death is documented. Box notes Q1, median, and Q3; whiskers show minimum and maximum. Symbols, closed circles, individual mice; open circles, means±SEM.

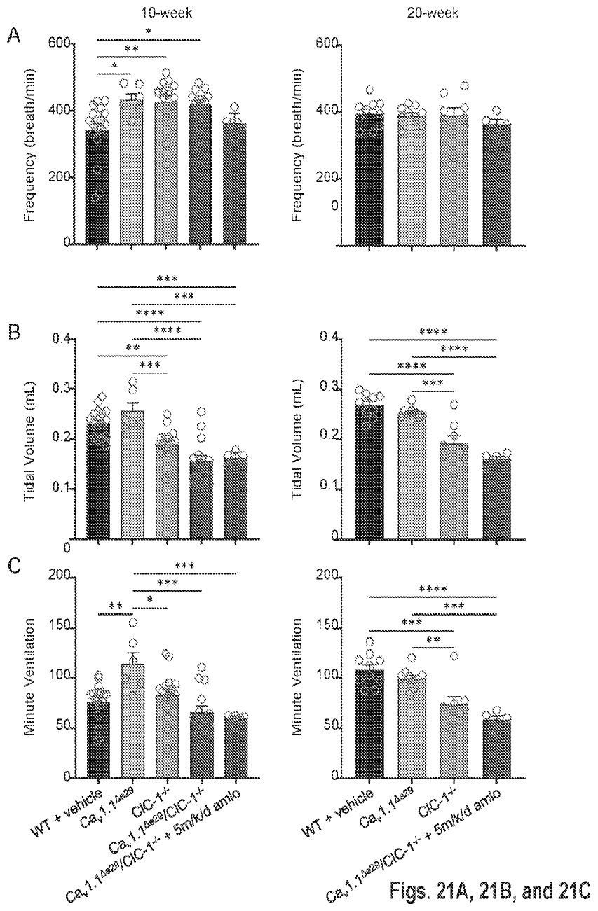

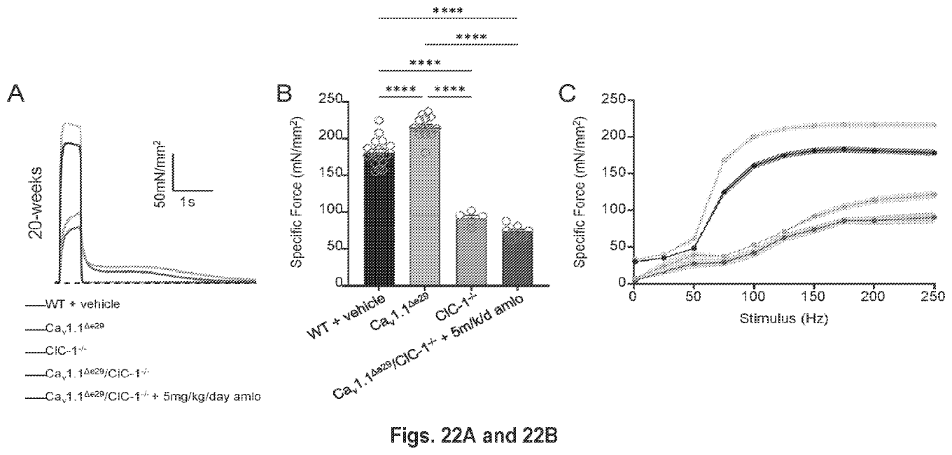

Next, the effects of amlodipine in improving respiration in Caᵥ1.1^{Δe29}/ClC-1^{-/-} mice was investigated. As shown in FIG. 21, Caᵥ1.1^{Δe29}/ClC-1^{-/-} mice have altered respiration compared to WT, Caᵥ1.1^{Δe29}/ClC-1^{-/-} mice treated with amlodipine do not have a significantly different frequency or minute ventilation compared to WT. Whole-body plethysmography for 10-wk (left) and 20-wk (right) old mice in the indicated genotype and treatment groups. Frequency of respiration (breaths/min) (FIG. 21A), tidal volume of respiration (mL) (FIG. 21B), and minute ventilation (FIG. 21C) are shown. At 10-weeks, Caᵥ1.1^{Δe29}/ClC-1^{-/-} mice have significantly increased frequency compared to WT mice. At this age, with 5 mg/kg/day amlodipine treatment in Caᵥ1.1^{Δe29}/ClC-1^{-/-} mice, there is a reduction in the frequency, and though there is not a significant difference that the untreated Caᵥ1.1^{Δe29}/ClC-1^{-/-} mice, there is also not a significant difference compared to WT frequencies. This would owe to a moderate impact that amlodipine treatment is having on frequency. Tidal volume and minute ventilation in 10-week treated mice are not improved compared to untreated Caᵥ1.1^{Δe29}/CC-1^{-/-} mice. At 20-weeks of age, the frequency of Caᵥ1.1^{Δe29}/ClC-1^{-/-} mice is not significantly different than that of WT mice. However, we do see a significant reduction in tidal volume and minute ventilation in Caᵥ1.1^{Δe29}/ClC-1^{-/-} mice compared to WT. Ultimately, respiratory function is being moderately impacted by amlodipine treatment in Caᵥ1.1^{Δe29}/ClC-1^{-/-} mice. Symbols, open circles, individual mice; bars and closed circles, means SEM. Note: Untreated Caᵥ1.1^{Δe29}/ClC-1^{-/-} mice do not survive to 20-week of age The effects of amlodipine in improving muscle function in Caᵥ1.1^{Δe29}/ClC-1^{-/-} mice was investigated. As shown in FIG. 22, EDL isolated from Caᵥ1.1^{Δe29}/ClC-1^{-/-} mice treated with amlodipine have similar muscle function to untreated ClC-1^{-/-} alone mice. FIG. 22A shows representative specific force traces elicited 150 Hz (500 ms) tetanic (right) stimulation of EDL muscle isolated from 20-wk mice. FIG. 22B shows average tetanic force generated during a 500 ms, 150 Hz tetanic stimulation in EDL muscle isolated from 20-week mice. FIG. 22C shows a plot of average stimulation frequency dependence of specific force generation from isolated EDL muscles at 20-week of age in the indicated genotype and treatment groups. At 20-weeks-of-age, there is not a significant impact on muscle function and muscle force generation in EDL that can be owed to amlodipine treatment. The reduction in force generation at 150 Hz and in the frequency dependence protocol in Caᵥ1.1^{Δe29}/ClC-1^{-/-} mice is comparable to the force deficits we see in ClC-1^{-/-} alone mice. Symbols, closed circles, means±SEM.

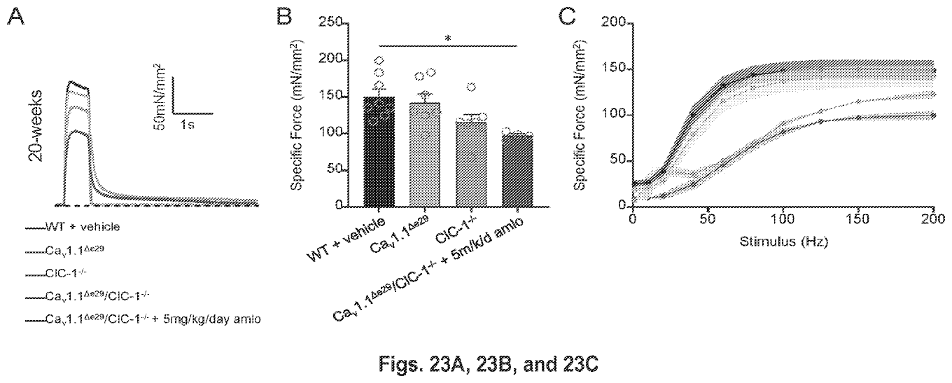

The effects of amlodipine in improving muscle function in diaphragm strip isolated from Caᵥ1.1^{Δe29}/ClC-1^{-/-} mice was also investigated. As shown in FIG. 23, amlodipine treatment improves muscle function in diaphragm strip isolated from Caᵥ1.1^{Δe29}/ClC-1^{-/-} mice treated with amlodipine has a force generation reduction compared to WT diaphragm. FIG. 23A shows representative specific force traces elicited 150 Hz (500 ms) tetanic (right) stimulation of diaphragm strip isolated from 20-wk mice. FIG. 23B shows average tetanic force generated during a 500 ms, 150 Hz tetanic stimulation in diaphragm strip isolated from 20-week mice. FIG. 23C shows a plot of average stimulation frequency dependence of specific force generation from isolated diaphragm strip at 20-week of age in the indicated genotype and treatment groups. At 20-weeks-of-age, there is not a significant impact on muscle function and muscle force generation in diaphragm that can be owed to amlodipine treatment. The reduction in force generation at 150 Hz and in the frequency dependence protocol in Caᵥ1.1^{Δe29}/ClC-1^{-/-} mice is comparable to the force deficits we see in ClC-1^{-/-} alone mice. To relate this back to FIG. 3, the frequency, tidal volume, and minute ventilation in treated Caᵥ1.1^{Δe29}/ClC-1^{-/-} mice compared to ClC-1^{-/-} alone mice showed no significant differences. It makes sense that we see similar values in diaphragm muscle function from the in vitro contraction experiments. Symbols, closed circles, means±SEM.

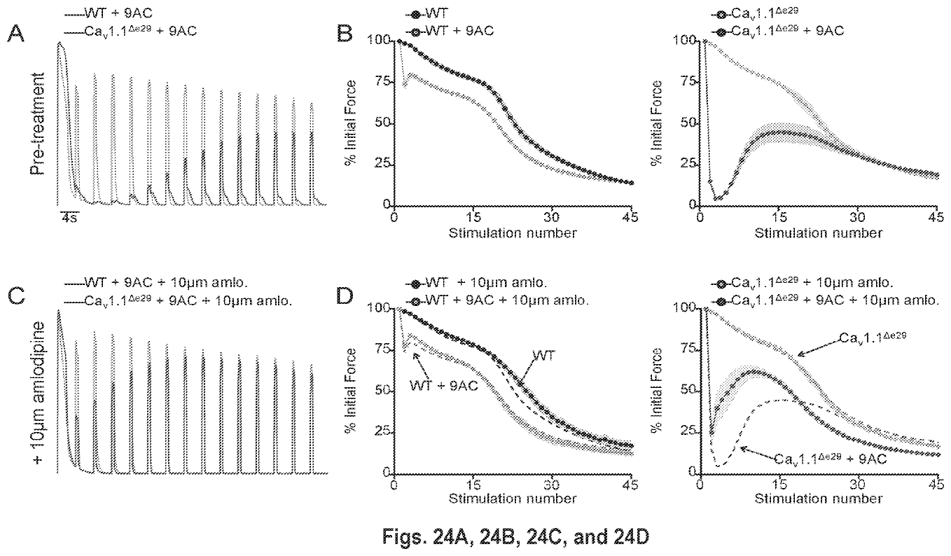

The effects of amlodipine in improving transient weakness in Caᵥ1.1^{Δe29}/ClC-1^{-/-} mice was also investigated. As shown in FIG. 24B, Caᵥ1.1^{Δe29} exacerbates transient weakness in myotonic muscle and is alleviated by amlodipine. FIG. 24A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). When myotonia is pharmacologically induced, we see a large drop in force generation in the Caᵥ1.1^{Δe29} EDL compared to WT. This is due to a more severe transient weakness in the myotonic Caᵥ1.1^{Δe29} tissue. FIG. 24B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. We further see the significant transient weakness in the myotonic Caᵥ1.1^{Δe29} EDL from the average data that was capture in the representative traces. There is only a slight transient weakness that is seen in the myotonic WT EDLs, and when myotonia is absent, there is no transient weakness seen for either WT or Caᵥ1.1^{Δe29} EDLs. FIG. 24C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM amlodipine (FIG. 24C) added to the bath. FIG. 24D shows a plot of the average peak tetanic EDL forces normalized to the initial stimulus, elicited by 44 subsequent 100 Hz, 500 ms tetanic stimulations separated by four seconds from WT+10 mM amlodipine (n=5), WT+9-AC+10 mM amlodipine (n=5), Caᵥ1.1^{Δe29}+10 mM amlodipine (n=5) and Caᵥ1.1^{Δe29}+9-AC+10 mM amlodipine e (n=5) EDLs. Dashed lines in FIG. 24D represent average data presented in FIG. 24B as a reference for pre-treatment. From both the representative traces as well as the average data, there is an improvement to transient weakness with 10 mm amlodipine treatment in the bath in the myotonic Caᵥ1.1^{Δe29} EDLs. The initial force generation drop is improved and the transient weakness in significantly improved during the recovery from transient weakness in the myotonic Caᵥ1.1^{Δe29} EDLs. Symbols, closed circles, mean±SEM. Note: Contralateral EDLs were used when possible.

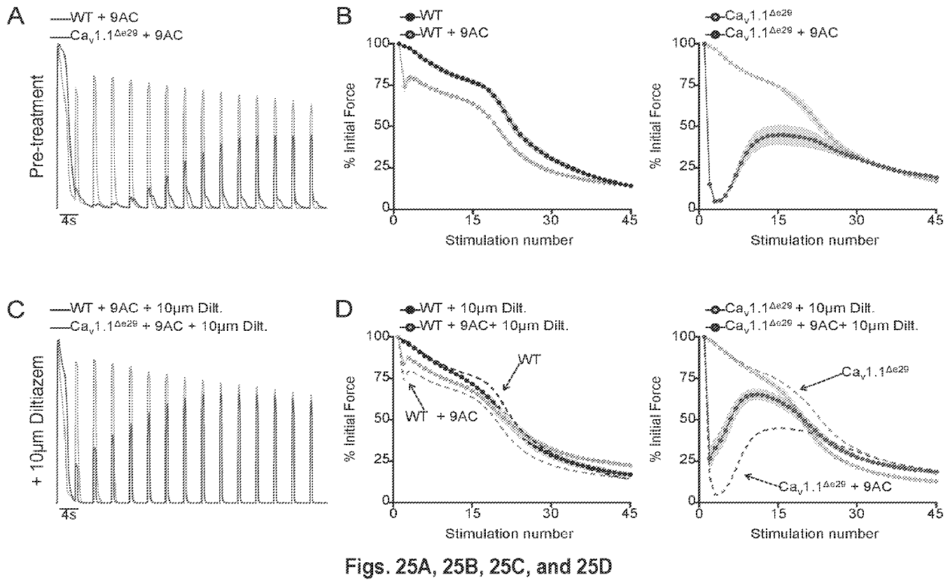

Next, the effects of amlodipine in improving transient weakness in myotonic muscle was investigated. As shown in FIG. 25, Caᵥ1.1^{Δe29} exacerbates transient weakness in myotonic muscle and is alleviated by diltiazem. FIG. 25A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 25B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. FIG. 25C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM diltiazem (FIG. 25C) added to the bath. FIG. 25D shows a plot of the average peak tetanic EDL forces normalized to the initial stimulus, elicited by 44 subsequent 100 Hz, 500 ms tetanic stimulations separated by four seconds from WT+10 mM diltiazem (n=5), WT+9-AC+10 mM diltiazem (n=5), Caᵥ1.1^{Δe29}+10 mM diltiazem (n=5) and Caᵥ1.1^{Δe29}+9-AC+10 mM diltiazem e (n=5) EDLs. Dashed lines in (FIG. 25D) represent average data presented in (FIG. 25B) as a reference for pre-treatment. Overall, we see a significant improvement in transient weakness in myotonic Caᵥ1.1^{Δe29} EDLs with diltiazem present in the bath. The initial drop in force generation between the first and second tetani as well as the overall recovery from transient weakness is improved with diltiazem. Symbols, closed circles, mean±SEM. Note: Contralateral EDLs were used when possible.

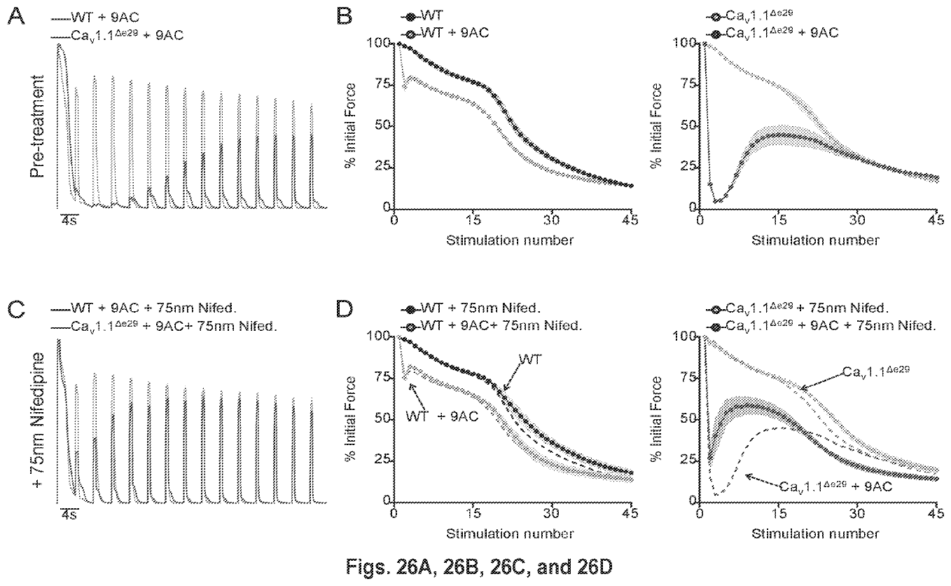

This example further provides the study of effects of nifedipine in muscle functions. As shown in FIG. 26, Caᵥ1.1^{Δe29} exacerbates transient weakness in myotonic muscle and is alleviated by nifedipine. FIG. 26A shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC added to the bath (pre-treatment). FIG. 26B shows a plot of the average peak tetanic EDL forces normalized to the initial stimulus, elicited by 44 subsequent 100 Hz, 500 ms tetanic stimulations separated by four seconds from WT (n=5), WT+9-AC (n=5), Caᵥ1.1^{Δe29} (n=5) and Caᵥ1.1^{Δe29}+9-AC (n=5) mice. FIG. 26C shows normalized representative force traces of the first 15 tetani (100 Hz, 500 ms) separated by four seconds, recorded ex vivo from EDLs isolated from 20-wk WT and Caᵥ1.1^{Δe29} mice in the presence of 100 mM 9-AC and 10 mM nifedipine (FIG. 26C) added to the bath. FIG. 26D shows a plot of the average peak tetanic EDL forces normalized to the initial stimulus, elicited by 44 subsequent 100 Hz, 500 ms tetanic stimulations separated by four seconds from WT+10 mM nifedipine (n=5), WT+9-AC+10 mM nifedipine (n=5), Caᵥ1.1^{Δe29}+10 mM nifedipine (n=5) and Caᵥ1.1^{Δe29}+9-AC+10 mM nifedipine e (n=5) EDLs. Dashed lines in FIG. 26D represent average data presented in FIG. 26B as a reference for pre-treatment. Overall, we see a significant improvement in transient weakness in myotonic Caᵥ1.1^{Δe29} EDLs with nifedipine present in the bath. The initial drop in force generation between the first and second tetani as well as the overall recovery from transient weakness is improved with nifedipine. Symbols, closed circles, mean±SEM. Note: Contralateral EDLs were used when possible.

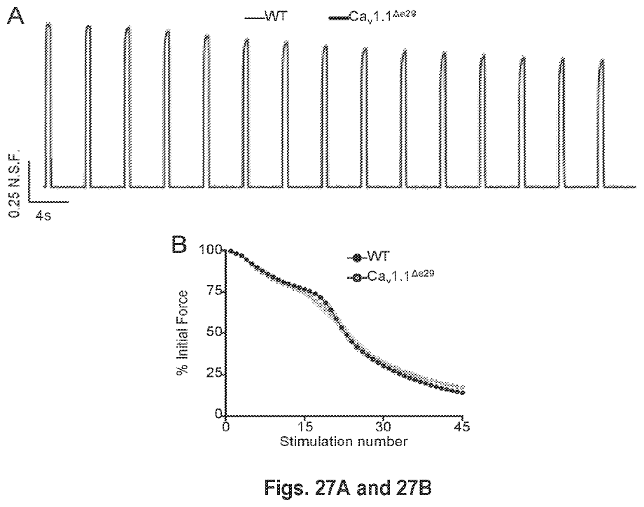

FIGS. 27A and 27B are a set of graphs showing that transient weakness is absent in WT and Caᵥ1.1^{Δe29} muscle. FIG. 27A shows representative traces of the first 15, tetani (150 Hz, 500 ms) separated by 4 seconds in 20-wk WT and Caᵥ1.1^{Δe29} EDL muscle. FIG. 27B shows a plot of the average peak tetanic EDL forces normalized to the initial stimulus, elicited by 44 subsequent 100 Hz, 500 ms tetanic stimulations separated by four seconds from EDLs isolated from 20-wk WT and Caᵥ1.1^{Δe29} mice. This was a control experiment to test if Caᵥ1.1^{Δe29} had an impact on transient weakness when no drugs are present in the bath. No difference between WT and Caᵥ1.1^{Δe29} EDLs was observed when 9AC is absent from the bath. Symbols, closed circles, mean±SEM.

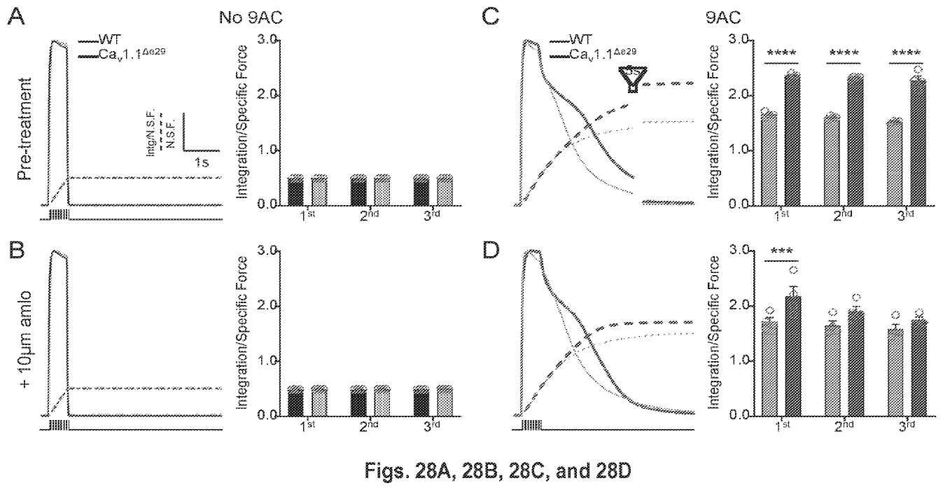

FIGS. 28A, 28B, 28C, and 28D are a set of graphs showing that Caᵥ1.1^{Δe29} significantly exacerbates myotonia, amlodipine reduced myotonia in myotonic WT and Caᵥ1.1^{Δe29} EDL. FIGS. 28A and 28B (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 28A, left) and presence of 10 mM amlodipine (FIG. 28B, left) added to the bath. Dashed lines represent accumulated force production. FIGS. 28A and 28B (right) show a plot of average integration normalized to specific force depicted in respective left panels. When there is no 9AC in the bath, WT and Caᵥ1.1^{Δe29} EDLs behave near identical to one another and the presence on amlodipine in the bath in B) has no affect with myotonia is not being induce by 9AC. FIGS. 28C and 28D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 28C, left) and presence of 10 mM amlodipine (FIG. 28D, left) added to the bath. Dashed lines represent accumulated force production. FIGS. 28C and 28D (right) show a plot of average integration normalized to specific force depicted in respective left panels. From the untreated experiment in FIG. 28C, there is a significantly prolonged myotonia in Caᵥ1.1^{Δe29} compared to WT EDL. The representative trace shows a myotonia of around 10 seconds in the Caᵥ1.1^{Δe29} EDL when 9AC is present. The average data to the right in FIG. 28C further shows this. In FIG. 28D, there is a reduction in the myotonia in the Caᵥ1.1^{Δe29} EDL when amlodipine is present in the bath. From the average data to the right in FIG. 28D, there is a significant difference between the first tetanus between WT and Caᵥ1.1^{Δe29}, but the following tetani show no difference in the myotonia with amlodipine treatment. Symbols, open circles, individual mice; bars, mean and SEM. Note: Contralateral EDLs were used when possible.

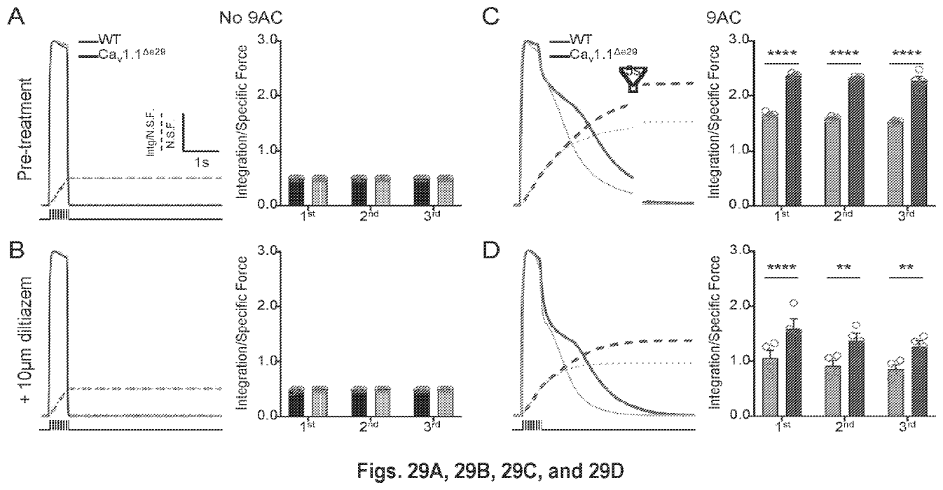

FIGS. 29A, 29B, 29C, and 29D are a set of graphs showing that Caᵥ1.1^{Δe29} significantly exacerbates myotonia, diltiazem reduced myotonia in myotonic WT, and Caᵥ1.1^{Δe29} EDL. FIGS. 29A and 29B (left) show normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 29A, left) and presence of 10 mM diltiazem (FIG. 29B, left) added to the bath. Dashed lines represent accumulated force production. FIGS. 29A and 29B (right) show a plot of average integration normalized to specific force depicted in respective left panels. FIGS. 29C and 29D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 29C, left) and presence of 10 mM diltiazem (FIG. 29D, left) added to the bath. Dashed lines represent accumulated force production. FIGS. 29C and 29D, right show a plot of average integration normalized to specific force depicted in respective left panels. In FIGS. 29D, there is a reduction in the myotonia in both the WT and the Caᵥ1.1^{Δe29} EDL when diltiazem is present in the bath. From the average data to the right in FIG. 29D we see that there is a significant difference between the tetanus between WT and Caᵥ1.1^{Δe29} when diltiazem is present, and for both WT and Caᵥ1.1^{Δe29} we see a significant reduction in myotonia when diltiazem is present in the bath compared to in FIG. 29C (right) for all 3 tetani. Symbols, open circles, individual mice; bars, mean, and SEM. Note: Contralateral EDLs were used when possible.

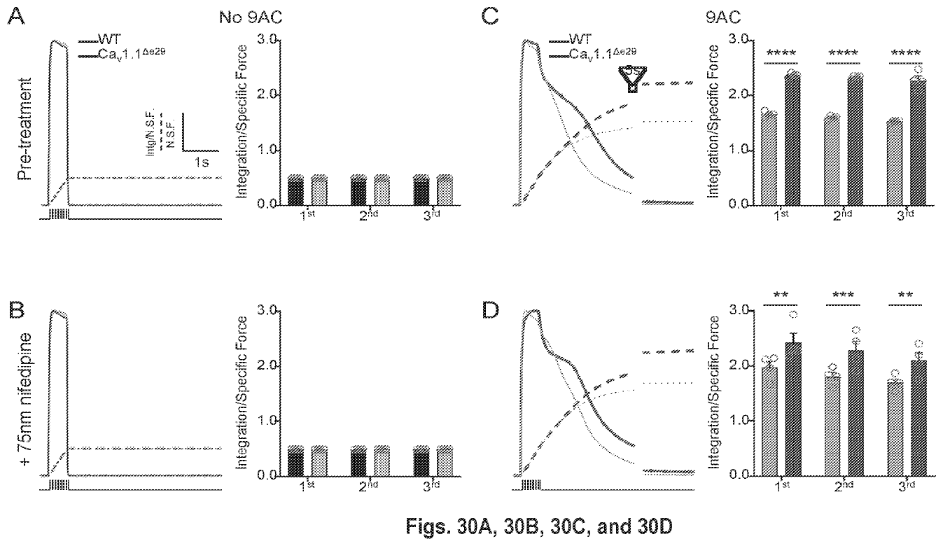

FIGS. 30A, 30B, 30C, and 30D are a set of graphs showing that Caᵥ1.1^{Δe29} significantly exacerbates myotonia, nifedipine reduced myotonia in myotonic WT and Caᵥ1.1^{Δe29} EDL. FIGS. 30A and B, left) Normalized representative force traces of the first of three tetani (100 Hz, 500 ms) separated by 3 minutes, recorded ex vivo from EDLs isolated from 20-wk WT and Caᵥ1.1^{Δe29} mice in the absence (pre-treatment) (FIG. 30A, left) and presence of 75 nM nifedipine (FIG. 30B, left) added to the bath. Dashed lines represent accumulated force production. FIGS. 30A and 30B (right) show a plot of average integration normalized to specific force depicted in respective left panels. FIGS. 30C and 30D (left) show normalized representative force traces of the first of three tetani (100 Hz, 500 ms) separated by 3 minutes, recorded ex vivo from EDLs incubated with 100 mM 9-AC, isolated from 20-wk WT and Caᵥ1.1^{Δe29} mice in the absence (pre-treatment) (FIG. 30C, left) and presence of 75 nM nifedipine (FIG. 30D, left) added to the bath. Dashed lines represent accumulated force production. FIGS. 30C and 30D (right) show a plot of average integration normalized to specific force depicted in respective left panels. In FIG. 30D, there is a reduction in the myotonia in the Caᵥ1.1^{Δe29} EDL when nifedipine is present in the bath. From the average data to the right in FIG. 30D, there is a significant difference in myotonia for each tetani comparing WT and Caᵥ1.1^{Δe29}. Symbols, open circles, individual mice; bars, mean and SEM. Note: Contralateral EDLs were used when possible.

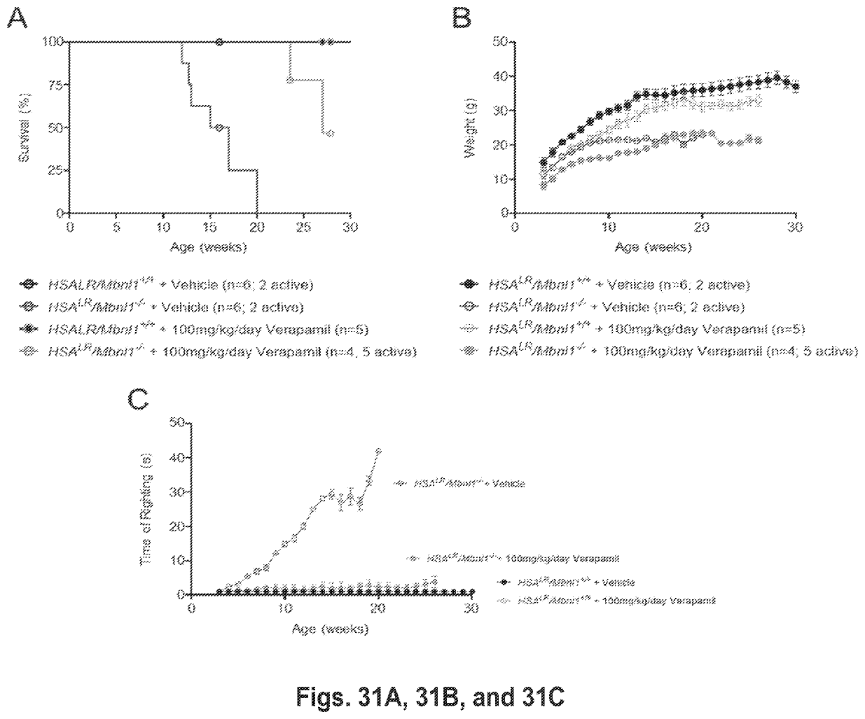

FIGS. 31A, 31B, and 31C are a set of graphs showing that the combination of HSA^{LR} with Mbnl1^{-/-} exhibit synthetic lethality, and result in significantly reduced survival and body weight, and prolonged time of righting. Verapamil treatment significantly improved survival and time of righting. FIG. 31A shows the results of Kaplan-Meier survival analysis of HSA^{LR}/Mbnl1^{+/+}+vehicle, HSA^{LR}/Mbnl1^{+/+}+100 mg/kg/day verapamil, HSA^{LR}/Mbnl^{-/-}+vehicle, and HSA^{LR}/Mbnl1^{-/-}+100 mg/kg/day verapamil. Untreated HSA^{LR}/Mbnl1^{-/-} mice have severely reduced lifespans. The treatment of 100 mg/kg/day verapamil significantly improves survival. FIG. 31B shows average week-to-week body weight change. Untreated HSA^{LR}/Mbnl1^{-/-} mice show a significantly reduced body weight from that of Untreated HSA^{LR}/Mbnl1^{+/+} mice. With verapamil treatment, weight in HSA^{LR}/Mbnl1^{-/-} mice is like that of untreated, but there is an increase in body weight as the mice age. FIG. 31C shows weekly time of righting reflex analysis of HSA^{LR}/Mbnl1^{+/+}+vehicle, HSA^{LR}/Mbnl1^{+/+}+100 mg/kg/day verapamil, HSA^{LR}/Mbnl1^{-/-}+vehicle, and HSA^{LR}/Mbnl1^{-/-}+100 mg/kg/day verapamil mice. Untreated HSA^{LR}/Mbnl1^{-/-} mice have significantly longer time if righting reflex. As they age, the time to right increases until they reach failure to right. This is when the mouse takes longer than 60 seconds to right. Treatment with 100 mg/kg/day verapamil in HSA^{LR}/Mbnl1^{-/-} mice significantly improves time of righting. Symbols, closed circles and error, means±SEM.

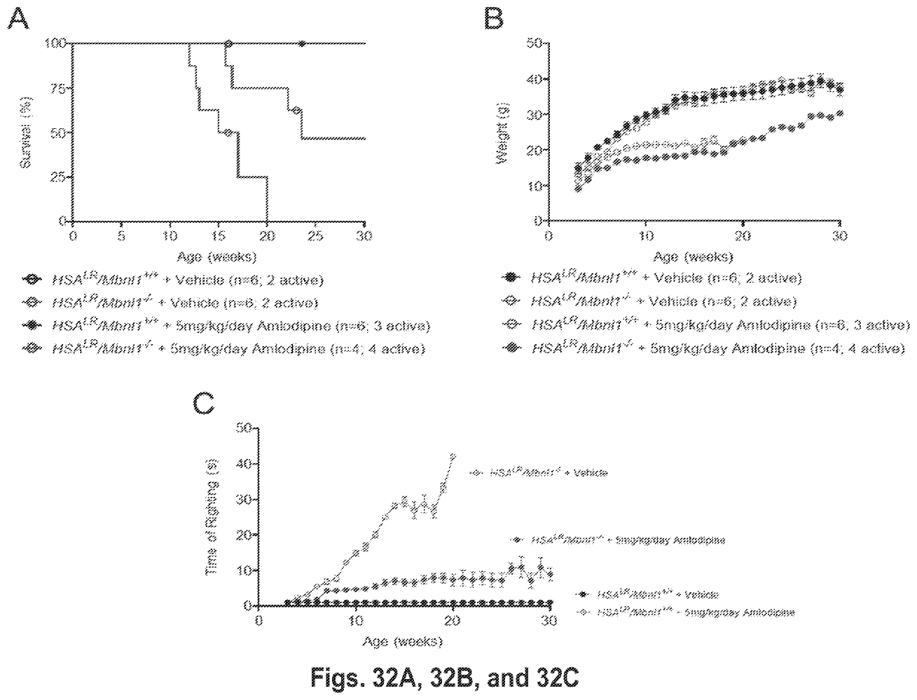

FIGS. 32A, 32B, and 32C are a set of graphs showing that the combination of HSA^{LR} with Mbnl1^{-/-} exhibit synthetic lethality, and result in significantly reduced survival and body weight, and prolonged time of righting. Amlodipine treatment significantly improved survival and time of righting. FIG. 32A shows the results of Kaplan-Meier survival analysis of HSA^{LR}/Mbnl1^{+/+}+vehicle, HSA^{LR}/Mbnl1^{+/+}+100 mg/kg/day amlodipine, HSA^{LR}/Mbnl^{-/-}+vehicle, and HSA^{LR}/Mbnl1^{-/-}+100 mg/kg/day amlodipine. Untreated HSA^{LR}/Mbnl1^{-/-} mice have severely reduced lifespans. The treatment of 100 mg/kg/day amlodipine significantly improves survival. FIG. 32B shows average week-to-week body weight change. Untreated HSA^{LR}/Mbnl1^{-/-} mice show a significantly reduced body weight from that of Untreated HSA^{LR}/Mbnl1^{+/+} mice. With amlodipine treatment, weight in HSA^{LR}/Mbnl1^{-/-} mice is like that of untreated, but there is an increase in body weight as the mice age. FIG. 32B shows weekly time of righting reflex analysis of HSA^{LR}/Mbnl1^{+/+}+vehicle, HSA^{LR}/Mbnl1^{+/+}+100 mg/kg/day amlodipine, HSA^{LR}/Mbnl^{-/-}+vehicle, and HSA^{LR}/Mbnl1^{-/-}+100 mg/kg/day amlodipine mice. Untreated HSA^{LR}/Mbnl1^{-/-} mice have significantly longer time if righting reflex. As they age, the time to right increases until they reach failure to right. This is when the mouse takes longer than 60 seconds to right. Treatment with 100 mg/kg/day amlodipine in HSA^{LR}/Mbnl1^{-/-} mice significantly improves time of righting. Symbols, closed circles and error, means±SEM. Statistical Analysis: One-way or Two-way ANOVA with multiple comparisons were used to test for significant differences between each of the groups tested. For survival, each group was compared to each other group Kaplan-Meier log-rank test. For all figures asterisks note p-values as follows, *P<0.05, **P<0.01, ***P<0.001, and ****P<0.0001 was used.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method of treating a myotonic disorder in a subject in need thereof, comprising administering to the subject a therapeutically effective amount of an agent that inhibits a calcium channel in a cell of the subject.

2. The method of claim 1, wherein the calcium channel is a Cav1.1 calcium channel.

3. The method of claim 1, wherein the agent is selected from the group consisting of verapamil, amlodipine, diltiazem, nifedipine, a stereoisomer thereof, a derivative thereof or a pharmaceutically acceptable salt thereof, and a combination thereof.

4. The method of claim 1, wherein the myotonic disorder is myotonic dystrophy, autosomal dominant or recessive myotonia congenita, or sodium channel myotonia.

5. The method of claim 4, wherein the myotonic dystrophy is myotonic dystrophy type 1 (DM1) or myotonic dystrophy type 2 (DM2).

6. The method of claim 4, wherein the myotonic dystrophy is associated with abnormal alternative splicing of RNA encoding CIC-1 chloride channel or Caᵥ1.1 calcium channel.

7. The method of claim 4, wherein the myotonia congenita is Becker or Thomsen myotonia congenita.

8. The method of claim 4, wherein the sodium channel myotonia is paramyotonia congentia, hyperkalemic periodic paralysis, or potassium-aggravated myotonia.

9. The method of claim 1, wherein the subject has one or more mutations in SCN4A, Clcn1, or Cac1ns, or has alternative splice isoforms of Clcn1 or Cac1ns.

10. The method of claim 1, wherein the subject is a mammal.

11. The method of claim 1, wherein the subject is a human.

12. The method of claim 1, wherein the agent is administered to the subject at one or more doses of from about 0.01 to about 100 mg/kg of body weight of the subject.

13. The method of claim 1, wherein the agent is administered at one or more doses of from about 1 to about 50 mg/kg of body weight of the subject.

14. The method of claim 1, wherein the agent is administered at one or more doses of from about 8 to about 16 mg/kg of body weight of the subject.

15. The method of claim 12, wherein the one or more doses of the agent are administered at least every 1 day, 3 days, 5 days, 1 week, 2 weeks, 3 weeks, or 4 weeks.

16. The method of claim 1, wherein the agent is administered to the subject intratumorally, intravenously, subcutaneously, intraosseously, orally, transdermally, sublingually, in sustained release, in controlled release, in delayed release, or as a suppository.

17. The method of claim 1, further comprising administering to the subject an additional therapeutic agent or therapy.

18. The method of claim 17, wherein the additional therapeutic agent is selected from the group consisting of ranolazine mexiletine, flecainide, tocainide, phenytoin, carbamazepine, lamotrigine, and a combination thereof.

19. The method of claim 1, wherein the treatment produces a therapeutic effect selected from the group consisting of reduced myotonia, reduced body weight loss, improved survival, improved muscle function, improved time of righting reflex, improved respiratory function, and improved diaphragm strength.

20. (canceled)

21. (canceled)

22. (canceled)

23. (canceled)

24. (canceled)

25. A pharmaceutical composition comprising: (a) a calcium channel blocker selected from the group consisting of verapamil, amlodipine, diltiazem, nifedipine, and a combination thereof, and (b) one or more agents selected from the group consisting of ranolazine mexiletine, flecainide, tocainide, phenytoin, carbamazepine, and lamotrigine.

26. (canceled)


SEQUENCE LISTING (2 sequences)

SEQ ID NO: 1 - DNA, 22 bp, synthetic construct
  source 1..22: mol_type = other DNA
ccagtcggaacagatgaaccac

SEQ ID NO: 2 - DNA, 22 bp, synthetic construct
  source 1..22: mol_type = other DNA
ccgatgaccgcgtagatgaaga